SEQUENCE LISTING (61 sequences)

SEQ ID NO: 1 - protein, 300 aa, Unknown
  Description of Unknown: Caspase-6 sequence
MASSASGLRRGHPAGGEENMTETDAFYKREMFDPAEKYKMDHRRRGIALIFNHERFFWHLTLPERRGTCADRDNLTRRFSDLGFEVKCFNDLKAEELLLKIHEVSTVSHADADCFVCVFLSHGEGNHIYAYDAKIEIQTLTGLFKGDKCHSLVGKPKIFIIQAARGNQHDVPVIPLDVVDNQTEKLDTNITEVDAASVYTLPAGADFLMCYSVAEGYYSHRETVNGSWYIQDLCEMLGKYGSSLEFTELLTLVNRKVSQRRVDFCKDPSAIGKKQVPCFASMLTKKLHFFPKSNLEHHHH

SEQ ID NO: 2 - protein, 95 aa, Unknown
  Description of Unknown: XBIR3 sequence
STNLPRNPSMADYEARIFTFGTWIYSVNKEQLARAGFYALGEGDKVKCFHCGGGLTDWKPSEDPWEQHAKWYPGCKYLLEQKGQEYINNIHLTHS

SEQ ID NO: 3 - protein, 118 aa, Artificial Sequence
  Description of Artificial Sequence: Synthetic polypeptide
MGSSHHHHHHSSGLVPRGSHMSTNTCLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 4 - protein, 6 aa, Artificial Sequence
  Description of Artificial Sequence: Synthetic 6xHis tag
HHHHHH

SEQ ID NO: 5 - protein, 117 aa, Artificial Sequence
  Description of Artificial Sequence: Synthetic polypeptide
MGSSHHHHHHSSGLVPRGSHMSTNTLPRNPSMADYEARIFTFGTWIYSVNKEQLARAGFYTDWALGEGDKVKCFHCGGGLRPSEDPWEQHARWYPGCRYLLEQRGQEYINNIHLTHS

SEQ ID NO: 6 - protein, 68 aa, Homo sapiens
EEARLKSFQNWPDYAHLTPRELASAGLYYTGIGDQVQCFCCGGKLKNWEPCDRAWSEHRRHFPNCFFV

SEQ ID NO: 7 - protein, 16 aa, Drosophila sp.
RQIKIWFQNRRMKWKK

SEQ ID NO: 8 - protein, 16 aa, Artificial Sequence
  Description of Artificial Sequence: Synthetic peptide
RRLRRLLRRLLRRLRR

SEQ ID NO: 9 - protein, 12 aa, Rabies lyssavirus
RVGRRRRRRRRR

SEQ ID NO: 10 - protein, 27 aa, Artificial Sequence
  Description of Artificial Sequence: Synthetic peptide
GWTLNSAGYLLGKINLKALAALAKKIL

SEQ ID NO: 11 - protein, 16 aa, Unknown
  Description of Unknown: pIsl sequence
PVIRVWFQNKRCKDKK

SEQ ID NO: 12 - protein, 13 aa, Human immunodeficiency virus
GRKKRRQRRRPPQ

SEQ ID NO: 13 - protein, 16 aa, Mus sp.
LLIILRRRIRKQAHAH

SEQ ID NO: 14 - protein, 27 aa, Unknown
  Description of Unknown: MPS sequence
GALFLGWLGAAGSTMGAWSQPKKKRKV

SEQ ID NO: 15 - protein, 19 aa, Artificial Sequence
  Description of Artificial Sequence: Synthetic peptide
VKRGLKLRHVRPRVTRMDV

SEQ ID NO: 16 - protein, 22 aa, Unknown
  Description of Unknown: M918 sequence
MVTVLFRRLRIRRACGPPRVRV

SEQ ID NO: 17 - protein, 22 aa, Unknown
  Description of Unknown: M1073 sequence
MVRRFLVTLRIRRACGPPRVRV

SEQ ID NO: 18 - protein, 28 aa, Bos sp.
MVKSKIGSWILVLFVAMWSDVGLCKKRP

SEQ ID NO: 19 - protein, 27 aa, Artificial Sequence
  Description of Artificial Sequence: Synthetic peptide
GALFLGFLGAAGSTMGAWSQPKKKRKV

SEQ ID NO: 20 - protein, 6 aa, Simian virus 40
KKKRKV

SEQ ID NO: 21 - protein, 17 aa, Human immunodeficiency virus
GALFLGFLGAAGSTMGA

SEQ ID NO: 22 - protein, 21 aa, Artificial Sequence
  Description of Artificial Sequence: Synthetic peptide
KETWWETWWTEWSQPKKKRKV

SEQ ID NO: 23 - protein, 12 aa, Artificial Sequence
  Description of Artificial Sequence: Synthetic peptide
KETWWETWWTEW

SEQ ID NO: 24 - protein, 18 aa, Artificial Sequence
  Description of Artificial Sequence: Synthetic peptide
KLALKLALKALKAALKLA

SEQ ID NO: 25 - protein, 18 aa, Artificial Sequence
  Description of Artificial Sequence: Synthetic peptide
LKTLTETLKELTKTLTEL

SEQ ID NO: 26 - protein, 18 aa, Artificial Sequence
  Description of Artificial Sequence: Synthetic peptide
QLALQLALQALQAALQLA

SEQ ID NO: 27 - protein, 30 aa, Artificial Sequence
  Description of Artificial Sequence: Synthetic polypeptide
WEAALAEALAEALAEHLAEALAEALEALAA

SEQ ID NO: 28 - protein, 28 aa, Unknown
  Description of Unknown: p28 sequence
LSTAADMQGVVTDGMASGLDKDYLKPDD

SEQ ID NO: 29 - protein, 12 aa, Hepatitis B virus
PLSSIFSRIGDP

SEQ ID NO: 30 - protein, 26 aa, Artificial Sequence
  Description of Artificial Sequence: Synthetic peptide
DPKGDPKGVTVTVTVTVTGKGDPKPD

SEQ ID NO: 31 - protein, 24 aa, Unknown
  Description of Unknown: Bactenecin-7 sequence
RRIRPRPPRLPRPRPRPLPFPRPG

SEQ ID NO: 32 - protein, 18 aa, Unknown
  Description of Unknown: SAP sequence
VRLPPPVRLPPPVRLPPP

SEQ ID NO: 33 - protein, 18 aa, Artificial Sequence
  Description of Artificial Sequence: Synthetic peptide
VELPPPVELPPPVELPPP

SEQ ID NO: 34 - protein, 10 aa, Crotalus durissus
CRWRWKCCKK

SEQ ID NO: 35 - protein, 20 aa, Herpes simplex virus 1
HGLASTLTRWAHYNALIRAF

SEQ ID NO: 36 - protein, 10 aa, Unknown
  Description of Unknown: FGF12 sequence
PIEVCMYREP

SEQ ID NO: 37 - protein, 17 aa, Artificial Sequence
  Description of Artificial Sequence: Synthetic peptide
CSIPPEVKFNKPFVYLI

SEQ ID NO: 38 - protein, 15 aa, Artificial Sequence
  Description of Artificial Sequence: Synthetic peptide
SDLWEMMMVSLACQY

SEQ ID NO: 39 - protein, 13 aa, Artificial Sequence
  Description of Artificial Sequence: Synthetic peptide
RLSGMNEVLSFRW

SEQ ID NO: 40 - protein, 17 aa, Artificial Sequence
  Description of Artificial Sequence: Synthetic peptide
  MOD_RES (1)..(1): Cys(NPys)
CRQIKIWFQNRRMKWKK

SEQ ID NO: 41-44 - skipped (4 entries)

SEQ ID NO: 45 - protein, 618 aa, Homo sapiens
MHKTASQRLFPGPSYQNIKSIMEDSTILSDWTNSNKQKMKYDFSCELYRMSTYSTFPAGVPVSERSLARAGFYYTGVNDKVKCFCCGLMLDNWKLGDSPIQKHKQLYPSCSFIQNLVSASLGSTSKNTSPMRNSFAHSLSPTLEHSSLFSGSYSSLSPNPLNSRAVEDISSSRTNPYSYAMSTEEARFLTYHMWPLTFLSPSELARAGFYYIGPGDRVACFACGGKLSNWEPKDDAMSEHRRHFPNCPFLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSTSDTTGEENADPPIIHFGPGESSSEDAVMMNTPVVKSALEMGFNRDLVKQTVQSKILTTGENYKTVNDIVSALLNAEDEKREEEKEKQAEEMASDDLSLIRKNRMALFQQLTCVLPILDNLLKANVINKQEHDIIKQKTQIPLQARELIDTILVKGNAAANIFKNCLKEIDSTLYKNLFVDKNMKYIPTEDVSGLSLEEQLRRLQEERTCKVCMDKEVSVVFIPCGHLVVCQECAPSLRKCPICRGIIKGTVRTFLS

SEQ ID NO: 46 - protein, 604 aa, Homo sapiens
MNIVENSIFLSNLMKSANTFELKYDLSCELYRMSTYSTFPAGVPVSERSLARAGFYYTGVNDKVKCFCCGLMLDNWKRGDSPTEKHKKLYPSCRFVQSLNSVNNLEATSQPTFPSSVTNSTHSLLPGTENSGYFRGSYSNSPSNPVNSRANQDFSALMRSSYHCAMNNENARLLTFQTWPLTFLSPTDLAKAGFYYIGPGDRVACFACGGKLSNWEPKDNAMSEHLRHFPKCPFIENQLQDTSRYTVSNLSMQTHAARFKTFFNWPSSVLVNPEQLASAGFYYVGNSDDVKCFCCDGGLRCWESGDDPWVQHAKWFPRCEYLIRIKGQEFIRQVQASYPHLLEQLLSTSDSPGDENAESSIIHFEPGEDHSEDAIMMNTPVINAAVEMGFSRSLVKQTVQRKILATGENYRLVNDLVLDLLNAEDEIREEERERATEEKESNDLLLIRKNRMALFQHLTCVIPILDSLLTAGIINEQEHDVIKQKTQTSLQARELIDTILVKGNIAATVFRNSLQEAEAVLYEHLFVQQDIKYIPTEDVSDLPVEEQLRRLQEERTCKVCMDKEVSIVFIPCGHLVVCKDCAPSLRKCPICRSTIKGTVRTFLS

SEQ ID NO: 47 - protein, 497 aa, Homo sapiens
MTFNSFEGSKTCVPADINKEEEFVEEFNRLKTFANFPSGSPVSASTLARAGFLYTGEGDTVRCFSCHAAVDRWQYGDSAVGRHRKVSPNCRFINGFYLENSATQSTNSGIQNGQYKVENYLGSRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSESDAVSSDRNFPNSTNLPRNPSMADYEARIFTFGTWIYSVNKEQLARAGFYALGEGDKVKCFHCGGGLTDWKPSEDPWEQHAKWYPGCKYLLEQKGQEYINNIHLTHSLEECLVRTTEKTPSLTRRIDDTIFQNPMVQEAIRMGFSFKDIKKIMEEKIQISGSNYKSLEVLVADLVNAQKDSMQDESSQTSLQKEISTEEQLRRLQEEKLCKICMDRNIAIVFVPCGHLVTCKQCAEAVDKCPMCYTVITFKQKIFMS

SEQ ID NO: 48 - protein, 1403 aa, Homo sapiens
MATQQKASDERISQFDHNLLPELSALLGLDAVQLAKELEEEEQKERAKMQKGYNSQMRSEAKRLKTFVTYEPYSSWIPQEMAAAGFYFTGVKSGIQCFCCSLILFGAGLTRLPIEDHKRFHPDCGFLLNKDVGNIAKYDIRVKNLKSRLRGGKMRYQEEEARLASFRNWPFYVQGISPCVLSEAGFVFTGKQDTVQCFSCGGCLGNWEEGDDPWKEHAKWFPKCEFLRSKKSSEEITQYIQSYKGFVDITGEHFVNSWVQRELPMASAYCNDSIFAYEELRLDSFKDWPRESAVGVAALAKAGLFYTGIKDIVQCFSCGGCLEKWQEGDDPLDDHTRCFPNCPFLQNMKSSAEVTPDLQSRGELCELLETTSESNLEDSIAVGPIVPEMAQGEAQWFQEAKNLNEQLRAAYTSASFRHMSLLDISSDLATDHLLGCDLSIASKHISKPVQEPLVLPEVFGNLNSVMCVEGEAGSGKTVLLKKIAFLWASGCCPLLNRFQLVFYLSLSSTRPDEGLASIICDQLLEKEGSVTEMCVRNIIQQLKNQVLFLLDDYKEICSIPQVIGKLIQKNHLSRTCLLIAVRTNRARDIRRYLETILEIKAFPFYNTVCILRKLFSHNMTRLRKFMVYFGKNQSLQKIQKTPLFVAAICAHWFQYPFDPSFDDVAVFKSYMERLSLRNKATAEILKATVSSCGELALKGFFSCCFEFNDDDLAEAGVDEDEDLTMCLMSKFTAQRLRPFYRFLSPAFQEFLAGMRLIELLDSDRQEHQDLGLYHLKQINSPMMTVSAYNNFLNYVSSLPSTKAGPKIVSHLLHLVDNKESLENISENDDYLKHQPEISLQMQLLRGLWQICPQAYFSMVSEHLLVLALKTAYQSNTVAACSPFVLQFLQGRTLTLGALNLQYFFDHPESLSLLRSIHFPIRGNKTSPRAHFSVLETCFDKSQVPTIDQDYASAFEPMNEWERNLAEKEDNVKSYMDMQRRASPDLSTGYWKLSPKQYKIPCLEVDVNDIDVVGQDMLEILMTVFSASQRIELHLNHSRGFIESIRPALELSKASVTKCSISKLELSAAEQELLLTLPSLESLEVSGTIQSQDQIFPNLDKFLCLKELSVDLEGNINVFSVIPEEFPNFHHMEKLLIQISAEYDPSKLVKLIQNSPNLHVFHLKCNFFSDFGSLMTMLVSCKKLTEIKFSDSFFQAVPFVASLPNFISLKILNLEGQQFPDEETSEKFAYILGSLSNLEELILPTGDGIYRVAKLIIQQCQQLHCLRVLSFFKTLNDDSVVEIAKVAISGGFQKLENLKLSINHKITEEGYRNFFQALDNMPNLQELDISRHFTECIKAQATTVKSLSQCVLRLPRLIRLNMLSWLLDADDIALLNVMKERHPQSKYLTILQKWILPFSPIIQK

SEQ ID NO: 49 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAEKVRRAIEQLAAMD

SEQ ID NO: 50 - protein, 1867 aa, Homo sapiens [sequence omitted]

SEQ ID NO: 51 - protein, 117 aa, Homo sapiens
RDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSE

SEQ ID NO: 52 - skipped

SEQ ID NO: 53 - protein, 96 aa, Unknown
  Description of Unknown: XBIR3 sequence
MSTNLPRNPSMADYEARIFTFGTWIYSVNKEQLARAGFYALGEGDKVKCFHCGGGLTDWKPSEDPWEQHAKWYPGCKYLLEQKGQEYINNIHLTHS

SEQ ID NO: 54 - protein, 69 aa, Unknown
  Description of Unknown: XBIR2 sequence
MEEARLKSFQNWPDYAHLTPRELASAGLYYTGIGDQVQCFCCGGKLKNWEPCDRAWSEHRRHFPNCFFV

SEQ ID NO: 55 - protein, 118 aa, Unknown
  Description of Unknown: BIR2 sequence
MRDHFALDRPSETHADYLLRTGQVVDISDTIYPRNPAMYSEEARLKSFQNWPDYAHLTPRELASAGLYYTGIGDQVQCFCCGGKLKNWEPCDRAWSEHRRHFPNCFFVLGRNLNIRSE

SEQ ID NO: 56 - protein, 12 aa, Artificial Sequence
  Description of Artificial Sequence: Synthetic peptide
  MISC_FEATURE (1)..(12): This sequence may encompass 3-12 residues
RRRRRRRRRRRR

SEQ ID NO: 57 - protein, 18 aa, Artificial Sequence
  Description of Artificial Sequence: Synthetic peptide
  MISC_FEATURE (1)..(18): This sequence may encompass 3-6 “Pro Pro Arg” repeating units
PPRPPRPPRPPRPPRPPR

SEQ ID NO: 58 - protein, 18 aa, Artificial Sequence
  Description of Artificial Sequence: Synthetic peptide
  MISC_FEATURE (1)..(18): This sequence may encompass 3-6 “Pro Arg Arg” repeating units
PRRPRRPRRPRRPRRPRR

SEQ ID NO: 59 - protein, 4 aa, Artificial Sequence
  Description of Artificial Sequence: Synthetic peptide
LEHD

SEQ ID NO: 60 - protein, 4 aa, Artificial Sequence
  Description of Artificial Sequence: Synthetic peptide
LEHD

SEQ ID NO: 61 - protein, 8 aa, Unknown
  Description of Unknown: RING finger domain sequence
CCCHCCCC

(19) United States
(12) Patent Application Publication
(10) Pub. No.: US 2022/0265767 A1
(43) Pub. Date: Aug. 25, 2022

(54) HIGH CONCENTRATION CELL PENETRATING CASPASE INHIBITOR CONJUGATES, COMPOSITIONS AND METHODS THEREOF

(71) Applicant: THE TRUSTEES OF COLUMBIA UNIVERSITY IN THE CITY OF NEW YORK, New York, NY (US)

(72) Inventors: Carol M. Troy, Hastings-on-Hudson, NY (US); Anna M. Potentski, New York, NY (US); Maria I. Avrutsky, Bronx, NY (US)

(21) Appl. No.: 17/736,946

(22) Filed: May 4, 2022

Related U.S. Application Data
(63) Continuation of application No. PCT/US2020/058683, filed on Nov. 3, 2020.
(60) Provisional application No. 62/930,371, filed on Nov. 4, 2019.

(51) Int. Cl. A61K 38/17; A61K 47/64; C07K 1/107

(52) U.S. Cl. CPC A61K 38/1761; C07K 1/1075; A61K 47/64

(57) ABSTRACT

A method of providing a high concentration disulfide-linked caspase inhibitor-cell penetrating peptide conjugate is described. The method includes incubating a caspase inhibitor having one or more thiol groups with a reducing agent selected from dithiothreitol (DTT), 2-mercaptoethanol (2-ME) and tris(2-carboxyethyl)phosphine (TCEP) to provide a reduced caspase inhibitor, removing the reducing agent from the reduced caspase inhibitor, and conjugating the reduced caspase inhibitor with a cell-penetrating peptide by a disulfide linkage.

### RELATED APPLICATIONS

This application is a Continuation of International Application No. PCT/US2020/058683 filed Nov. 3, 2020, which claims priority to U.S. Provisional Patent Application No. 62/930,371, filed Nov. 4, 2019, the disclosure of both of which is incorporated herein by reference in its entirety.

### STATEMENT OF GOVERNMENT INTEREST

This invention was made with government support under grant number NS081333 awarded by National Institutes of Health. The government has certain rights in the invention.

### SEQUENCE LISTING

The Sequence Listing associated with this application is provided in text format in lieu of a paper copy, and is hereby incorporated by reference into the specification. The name of the text file containing the Sequence Listing is 210271.410C1 SEQUENCE LISTING.txt. The text file is 65.4 KB, was created on May 4, 2022, and is being submitted electronically via EFS-Web.

### TECHNICAL FIELD

The present disclosure relates to formulation of high concentration cell-penetrating caspase inhibitor conjugates. The present disclosure also relates to compositions of the high concentration cell-penetrating caspase inhibitors, and methods of using the high concentration cell-penetrating caspase inhibitors for the inhibition of apoptosis associated with ischemic injury in the central nervous system (“CNS”) and ameliorating neurodegenerative diseases associated with apoptosis in the central nervous system, inhibition of diabetic macular edema (DME) and/or retinal vein occlusion (RVO), and preventing or reducing inflammation.

### BACKGROUND

Cell-penetrating forms of caspase inhibitors have previously been shown to be efficacious in cell culture and in vivo rat and mouse studies to inhibit apoptosis associated with ischemic injury in the CNS (U.S. patent application Ser. No. 13/768.687, filed on Feb. 15, 2013, and published as U.S. Patent Application Publication No. US 2014/0024597), or inhibition of diabetic macular edema (DME) and/or retinal vein occlusion (RVO) (U.S. patent application Ser. No. 16/243,884, filed on Jan. 9, 2019, and published as U.S. Patent Application Publication No. US 2019/0142915), and preventing or reducing inflammation (U.S. Provisional Patent Application No. 62/840,234, filed on Apr. 29, 2019).

Administration of pharmaceutical compositions in larger animals and humans typically requires production of pharmaceutical compositions in higher concentrations to allow higher doses for improved efficacy. However, production of high concentration compositions of cell-penetrating forms of caspase inhibitors has proved challenging.

### SUMMARY

According to a first aspect, a method of providing a disulfide-linked caspase inhibitor-cell penetrating peptide conjugate is described. The method includes incubating a caspase inhibitor having one or more thiol groups with a reducing agent selected from dithiothreitol (DTT), 2-mercaptoethanol (2-ME) and tris(2-carboxyethyl)phosphine (TCEP) to provide a reduced caspase inhibitor. The method further includes removing the reducing agent from the reduced caspase inhibitor and conjugating the reduced caspase inhibitor with a cell-penetrating peptide by a disulfide linkage.

The method may further include any of the following elements in any combination, unless clearly mutually exclusive:

i) the caspase inhibitor may be selected from the group consisting of a caspase -1, -2, -3, -4, -5, -6, -7, -8, -9, -10, -11, -12, and -14 inhibitor;

ii) the caspase inhibitor may be selected from a XBIR3, a XBIR2, a linker-BIR2 and a dominant-negative caspase 6;

iii) the removing the reducing agent may be by filtration;

iv) the method may further include a buffer exchange wherein the reduced caspase inhibitor is comprised in a pharmaceutically acceptable excipient;

v) the cell-penetrating peptide may be selected from Penetratin1, transportan, pIsl, TAT(48-60), pVEC, MTS, MAP, polyarginines, DPV1047, M918, M1073, BPrPr (1-28), MPG, Pep-1, MAP12, MAP17, GALA, p28, PreS2, VT5, Bac 7 [Bac (1-24)], PPR, PRR, SAP, SAP(E), CyLoP-1, gH 625, CPP-C, C105Y, Pep-7, and SG3;

vi) the caspase inhibitor-cell penetrating peptide conjugate is a disulfide-linked

vii) the reduced caspase inhibitor may have no more than 40% caspase inhibitor dimers;

viii) the reduced caspase inhibitor may have at least 3-fold less dimer formation than the caspase inhibitor that has not been treated with the reducing agent; and

ix) the caspase inhibitor-cell penetrating peptide conjugate may have a concentration greater than 1 mM;

According to a second aspect, a composition including a disulfide linked caspase inhibitor-cell penetrating peptide conjugate and a pharmaceutically acceptable carrier is described. The caspase inhibitor-cell penetrating peptide conjugate has a concentration greater than 1 mM.

The composition may further include any of the following elements in any combination, unless clearly mutually exclusive:

i) the caspase inhibitor-cell penetrating peptide conjugate may include a caspase inhibitor selected from the group consisting of a caspase -1, -2, -3, -4, -5, -6, -7, -8, -9, -10, -11, -12, and -14 inhibitor;

ii) the caspase inhibitor is selected from a XBIR3, a XBIR2, a linker-BIR2 and a dominant-negative caspase 6;

iii) the cell-penetrating peptide may be selected from Penetratin1, transportan, pIsl, TAT(48-60), pVEC, MTS, MAP, polyarginines, DPV1047, M918, M1073, BPrPr (1-28), MPG, Pep-1, MAP12, MAP17, GALA, p28, PreS2, VT5, Bac 7 [Bac (1-24)], PPR, PRR, SAP, SAP(E), CyLoP-1, gH 625, CPP-C, C105Y, Pep-7, and SG3;

iv) the caspase inhibitor-cell penetrating peptide conjugate is a disulfide-linked Penetratin1-XBIR3; and

v) the composition may be formulated for injection, inhalation, or topical administration.

### BRIEF DESCRIPTION OF THE DRAWINGS

For a more complete understanding of the present disclosure and the associated features and advantages, reference is now made to the following description, taken in conjunction with the accompanying drawings, which are not to scale, and in which:

FIG. 1 is a schematic of an example procedure for reducing XBIR3 using dithiothreitol (DTT) as a reducing agent.

FIG. 2 is an image of an example PAGE (polyacrylamide gel electrophoresis) gel after PAGE separation of samples of linked Pen1-XBIR3, or unlinked XBIR3 with or without reducing XBIR3 with dithiothreitol (DTT). FIG. 2 shows linkage of up to 3.1 mM Pen1-XBIR3 using DTT-treated XBIR3.

FIG. 3 is an image of an example PAGE (polyacrylamide gel electrophoresis) gel after PAGE separation of samples of XBIR3 with or without DTT treatment, and linked Pent-XBIR3 produced with or without DTT treatment of the XBIR3. FIG. 3 shows linkage of up to 500 Pen1-XBIR3 using DTT-treated XBIR3.

FIG. 4A is Table summarizing example secondary structure results from circular dichroism analysis of XBIR3 at a concentration of 0.3 mg/ml and a temperature of 23° C.

FIG. 4B is a graph reporting example fitting results for the XBIR3 data reported in FIG. 4A.

FIG. 5A is Table summarizing example secondary structure results from circular dichroism analysis of Pent-XBIR3 at a concentration of 0.3 mg/ml and a temperature of 23° C.

FIG. 5B is a graph reporting example fitting results for the Pen1-XBIR3 data reported in FIG. 5A.

FIG. 6A is Table summarizing example secondary structure results from circular dichroism analysis of XBIR3 at a concentration of 0.3 mg/ml and a temperature of 4° C.

FIG. 6B is a graph reporting example fitting results for the XBIR3 data reported in FIG. 6A.

FIG. 7A is Table summarizing example secondary structure results from circular dichroism analysis of Pent-XBIR3 at a concentration of 0.3 mg/ml and a temperature of 4° C.

FIG. 7B is a graph reporting example fitting results for the Pen1-XBIR3 data reported in FIG. 7A.

FIG. 8A is Table summarizing example secondary structure results from circular dichroism analysis of XBIR3 at a concentration of 0.3 mg/ml and a temperature of 34° C.

FIG. 8B is a graph reporting example fitting results for the XBIR3 data reported in FIG. 8A.

FIG. 9A is Table summarizing example secondary structure results from circular dichroism analysis of Pent-XBIR3 at a concentration of 0.3 mg/ml and a temperature of 34° C.

FIG. 9B is a graph reporting example fitting results for the Pen1-XBIR3 data reported in FIG. 9A.

FIG. 10A is Table summarizing example secondary structure results from circular dichroism analysis of XBIR3 at a concentration of 0.2 mg/ml and a temperature of 23° C.

FIG. 10B is a graph reporting example fitting results for the XBIR3 data reported in FIG. 10A.

FIG. 11A is Table summarizing example secondary structure results from circular dichroism analysis of Pen1-XBIR3 at a concentration of 0.32 mg/ml and a temperature of 23° C.

FIG. 11B is a graph reporting example fitting results for the Pent-XBIR3 data reported in FIG. 11A.

FIG. 12A is Table summarizing example secondary structure results from circular dichroism analysis of XBIR3 at a concentration of 0.2 mg/ml and a temperature of 4° C.

FIG. 12B is a graph reporting example fitting results for the XBIR3 data reported in FIG. 12A.

FIG. 13A is Table summarizing example secondary structure results from circular dichroism analysis of Pent-XBIR3 at a concentration of 0.2 mg/ml and a temperature of 4° C.

FIG. 13B is a graph reporting example fitting results for the Pent-XBIR3 data reported in FIG. 13A.

FIG. 14A is Table summarizing example secondary structure results from circular dichroism analysis of XBIR3 at a concentration of 0.2 mg/ml and a temperature of 34° C.

FIG. 14B is a graph reporting example fitting results for the XBIR3 data reported in FIG. 14A.

FIG. 15A is Table summarizing example secondary structure results from circular dichroism analysis of Pent-XBIR3 at a concentration of 0.2 mg/ml and a temperature of 34° C.

FIG. 15B is a graph reporting example fitting results for the Pen1-XBIR3 data reported in FIG. 15A.

### DETAILED DESCRIPTION

The present disclosure generally relates to formulation of high concentration cell-penetrating caspase inhibitors. In certain embodiments, the cell-penetrating caspase inhibitors are useful for the inhibition of apoptosis associated with ischemic injury in the central nervous system (CNS), or ameliorating neurodegenerative diseases associated with apoptosis in the CNS, as described in U.S. patent application Ser. No. 13/768.687, filed on Feb. 15, 2013, and published as U.S. Patent Application Publication No. US 2014/0024597, the disclosure of which is hereby incorporated by reference in its entirety. In certain embodiments, the cell-penetrating caspase inhibitors are useful for preventing or reducing inflammation, as described in U.S. Provisional Patent Application No. 62/840,234, filed on Apr. 29, 2019, the disclosure of which is hereby incorporated by reference in its entirety. In certain embodiments, the cell-penetrating caspase inhibitors are useful for inhibition of diabetic macular edema (DME) and/or retinal vein occlusion (RVO), as described in U.S. patent application Ser. No. 16/243,884, filed on Jan. 9, 2019, and published as U.S. Patent Application Publication No. US 2019/0142915, the disclosure of which is hereby incorporated by reference in its entirety.

### Pro-Apoptotic Caspases in Ischemic Stroke and Neurodegenerative Diseases

Stroke is the third leading cause of death and the leading cause of motor disability in the industrialized world. In ischemic stroke, which accounts for 85% of all stroke cases, thrombosis or embolism leads to an occlusion of a major artery that supplies the brain with oxygen, and depletion of oxygen results in tissue injury. The injured territory downstream from the occlusion is comprised of an ischemic core and its surrounding penumbra. The ischemic core is the territory where perfusion decreased below the threshold for viability, and where the cells are both electrically silent and irreversibly injured. Injury to the core occurs primarily via necrosis, however, there is recent evidence arguing that apoptosis may also occur in the core. (Yuan, Apoptosis 14 (4), 469-477 (2009)). In contrast, the area defined as the penumbra continues to receive blood and nutrients, although at a reduced capacity, and these cells could potentially remain viable. When cell death occurs in the penumbra, it is thought to be due to apoptosis. (Ribe, et al., Biochem J 415 (2), 165-182 (2008)). With timely reperfusion, either spontaneous or therapeutic, this territory may be salvaged. However, restoration of blood flow can also induce ‘reperfusion injury’, which exacerbates inflammation, excitotoxicity, and apoptotic cell injury. (Ribe, et al, Biochem J 415 (2), 165-182 (2008)). In humans, apoptotic markers, including cleaved caspases, can be observed in the peri-infarct region from 24 hrs to 26 days following a stroke and diffusion tensor imaging reveals extensive loss of axonal tracts in the stroke penumbra. (Broughton, et al., Stroke 40 (5), e331-339 (2009); Mitsios, et al., Cell Biochem Biophys 47 (1), 73-86 (2007); Lie, et al., Stroke 35 (1), 86-92 (2004); Thomalla, et al., Neuroimage 22 (4), 1767-1774 (2004)).

As described in U.S. patent application Ser. No. 13/768,687, members of the caspase family of proteins (including caspases -1, -2, -3, -4, -5, -6, -7, -8, -9, 10, -11, -12, and -14) have been identified as apoptotic molecules that become activated following ischemic injury. Similarly, caspases such as caspase-6, among others, have been implicated in neuronal death in multiple neurodegenerative diseases. Accordingly, U.S. patent application Ser. No. 13/768,687 relates to compositions and methods for the inhibition of apoptosis associated with ischemic injury in the CNS, and also to compositions and methods for the inhibition of apoptosis associated with neurodegenerative diseases, such as such as Alzheimer's Disease, Mild Cognitive Impairment, Parkinson's Disease, amyotrophic lateral sclerosis, Huntington's disease, and Creutzfeld-Jacob disease, among others.

### Caspase-9 Signalling Pathway and Inflammation

Contrary to prior assumptions, the disclosure of U.S. Provisional Patent Application No. 62/840,234 establishes caspase-9 as an inflammatory caspase, and that inhibition of a caspase-9 signaling pathway can prevent or decrease inflammation.

As used herein, the term “inflammation” refers to part of a complex biological response of body tissues to harmful stimuli, such as pathogens, damaged cells, or irritants, among others, and may be, at least initially, a protective response, that may involve immune cells, blood vessels, and molecular mediators. In general, the function of inflammation is to eliminate the initial cause of cell injury, clear out necrotic cells and tissues damaged from the original insult and the inflammatory process, and initiate tissue repair. Symptoms of inflammation in a patient may include heat, pain, redness, swelling, and loss of function. Inflammation is generally considered to be a generic mechanism of innate immunity. This contrasts with adaptive immunity, which typically includes a specific immune response for a specific pathogen. In some cases, too little inflammation can lead to progressive tissue destruction by the harmful stimulus and compromise the survival of the patient. In contrast, chronic inflammation may lead to a host of diseases. Inflammation is therefore normally closely regulated by the body. Inflammation can be classified as either acute or chronic. Acute inflammation refers to the initial response of the body to harmful stimuli and involves increased movement of plasma and leukocytes from the blood into the injured tissues. A series of biochemical events propagates and matures the inflammatory response, involving the local vascular system, the immune system, and various cells within the injured tissue. Prolonged inflammation, referred to as chronic inflammation, leads to a progressive shift in the type of cells present at the site of inflammation, such as mononuclear cells, and may be characterized by simultaneous destruction and healing of the tissue from the inflammatory process.

Acute inflammation is typically a short-term process, usually appearing within a few minutes or hours and begins to cease upon the removal of the injurious stimulus. It involves a coordinated and systemic mobilization response locally of various immune, endocrine and neurological mediators of acute inflammation. In a normal healthy response, it becomes activated, clears the pathogen and begins a repair process and then ceases. The process of acute inflammation is typically initiated by resident immune cells already present in the involved tissue, such as resident macrophages, dendritic cells, histiocytes, Kupffer cells and mast cells. These cells possess surface receptors known as pattern recognition receptors (PRRs), which bind to and thereby recognize two subclasses of molecules: pathogen-associated molecular patterns (PAMPs) and damage-associated molecular patterns (DAMPs). PAMPs are compounds that are associated with various pathogens, but which are distinguishable from host molecules. DAMPs are compounds that are associated with host-related injury and cell damage. At the onset of an infection, burn, or other injuries, these cells undergo activation (e.g., one of the PRRs recognize a PAMP or DAMP) and release inflammatory mediators responsible for the clinical signs of inflammation. The mediator molecules also alter the blood vessels to permit the migration of leukocytes, mainly neutrophils and macrophages, outside of the blood vessels (referred to as extravasation) into the tissue.

In addition to cell-derived mediators, several biochemical cascade systems involving plasma proteins act in parallel to initiate and propagate the inflammatory response. These include without limitation, bradykinin, complement system proteins such as C3, C5a, C5b, C6, C7, C8 and C9, Factor XII, plasmin, and thrombin, among others. Acute inflammation can also involve the movement of plasma fluid containing proteins such as fibrin and immunoglobulins into inflamed tissue.

The cellular component of acute inflammation often involves leukocytes, which normally reside in blood and move into the inflamed tissue via extravasation to aid in inflammation. Some act as phagocytes, ingesting bacteria, viruses, and cellular debris. Others release enzymatic granules that damage pathogenic invaders. Leukocytes also release inflammatory mediators that develop and maintain the inflammatory response. In general, acute inflammation may be mediated by granulocytes, for example, whereas chronic inflammation may be mediated by mononuclear cells such as monocytes and lymphocytes. Cell derived-mediators of inflammation include, without limitation, lysosome granules, histamine, interferon-γ, interleukin-8, leukotriene B4, leukotriene C4, leukotriene D4, 5-oxo-eicosatetraenoic acid, 5-Hydroxyeicosatetraenoic acid, prostaglandins, nitric oxide, tumor necrosis factor alpha, interleukin-1, and tryptase, among others.

In some cases, specific patterns of acute and chronic inflammation are seen during particular situations that arise in the body. For example, granulomatous inflammation refers to the formation of granulomas, associated with diseases such as tuberculosis, leprosy, sarcoidosis, and syphilis. Fibrinous inflammation refers to inflammation resulting in a large increase in vascular permeability allows fibrin to pass through the blood vessels. If an appropriate pro-coagulative stimulus is present, such as cancer cells, a fibrinous exudate may be deposited. This is commonly seen in serous cavities, where the conversion of fibrinous exudate into a scar can occur between serous membranes, limiting their function. The deposit sometimes forms a pseudo-membrane sheet. During inflammation of the intestine (e.g., pseudomembranous colitis), pseudo-membranous tubes can be formed. Purulent inflammation refers to inflammation resulting in a large amount of pus, which may contain neutrophils, dead cells, and fluid. For example, infection by pyogenic bacteria such as staphylococci is characteristic of purulent inflammation. Large, localized collections of pus enclosed by surrounding tissues are referred to as abscesses. Serous inflammation refers to copious effusion of non-viscous serous fluid, commonly produced by mesothelial cells of serous membranes, but may be derived from blood plasma. Skin blisters exemplify serous inflammation. Ulcerative inflammation refers to inflammation occurring near an epithelium and can result in the necrotic loss of tissue from the surface, exposing lower layers. The subsequent excavation in the epithelium is known as an ulcer.

Inflammation can result from various causes, including, without limitation, physical causes such as burns, frostbite, blunt or penetrating injury, foreign bodies e.g. splinters, dirt and debris, trauma, ionizing radiation; biological causes such as infection by pathogens, immune reactions due to hypersensitivity, and stress; chemical causes such as chemical irritants, toxins, or alcohol, among other causes identifiable by skilled persons.

By convention, many types of inflammation are indicated by the suffix “-itis”, and are often associated with a particular tissue of a patient. For example, some types of inflammation include, without limitation, appendicitis, bronchitis, bursitis, colitis, cystitis, dermatitis, encephalitis, gingivitis, meningitis, myelitis, nephritis, neuritis, periodontitis, pharyngitis, phlebitis, prostatitis, pulmonitis, rhinitis, sinusitis, tendonitis, tonsillitis, urethritis, vaginitis, and vasculitis, among others described herein or identifiable by skilled persons upon reading the present disclosure.

The term “vasculitis” refers to a group of disorders that destroy blood vessels by inflammation. Arteries and/or veins may be affected in vasculitis. Without limitation to theory, vasculitis is often caused by leukocyte migration and resultant damage. Possible symptoms include, without limitation, general symptoms such as fever and weight loss, skin symptoms such as palpable purpura and livedo reticularis, muscle and joint symptoms such as myalgia, myositis, arthralgia and arthritis, nervous system symptoms such as mononeuritis multiplex, headache, stroke, tinnitus, reduced visual acuity, and acute visual loss, heart and artery symptoms such as myocardial infarction, hypertension, and gangrene, respiratory tract symptoms such as nose bleeds, bloody cough, and lung infiltrates gastrointestinal tract symptoms such as abdominal pain, bloody stool, and perforations, and kidney symptoms such as glomerulonephritis, among others. For example, vasculitis may include without limitation, cutaneous small-vessel vasculitis which may affect the skin and kidneys; granulomatosis with polyangiitis which may affect nose, lungs, and kidneys; eosinophilic granulomatosis with polyangiitis which may affect lungs, kidneys, heart, and skin; Behcet's disease which may affect sinuses, brain, eyes, skin, lungs, kidneys, and joints; Kawasaki disease which may affect skin, heart, mouth, and eyes; Buerger's disease which may affect leg arteries and veins; Takayasu's arteritis, polyarteritis nodosa and giant cell arteritis which may affect arteries. Some diseases have vasculitis as an accompanying feature, including, without limitation, rheumatic diseases, such as rheumatoid arthritis, systemic lupus erythematosus, and dermatomyositis; cancers, such as lymphomas; infections, such as hepatitis C. In addition, exposure to certain chemicals and drugs, such as amphetamines, cocaine, and anthrax vaccines which contain the Anthrax Protective Antigen as the primary ingredient may be associated with vasculitis. In pediatric patients, varicella inflammation may be followed by vasculitis of intracranial vessels. This condition is referred to as post varicella angiopathy and may be associated with arterial ischemic strokes in children. Vasculitis may be diagnosed in a patient using various methods known in the art. For example, laboratory tests of blood or body fluids may show signs of inflammation in the body, such as increased erythrocyte sedimentation rate (ESR), elevated C-reactive protein (CRP), anemia, increased white blood cell count and eosinophilia, among other signs. A biopsy of an affected organ or tissue, such as skin, sinuses, lung, nerve, brain and kidney, and so on, or an angiogram (x-ray assay of the blood vessels) may reveal the pattern of blood vessel inflammation. 18F-fluorodeoxyglucose positron emission tomography/computed tomography (FDG-PET/CT) imaging may be used, e.g. in patients with suspected Large Vessel Vasculitis, due to the enhanced glucose metabolism of inflamed vessel walls.

Endothelial inflammation can include inflammation of the cells that line the interior surface of blood vessels and lymphatic vessels. The endothelium refers to a thin layer of squamous cells called endothelial cells. Endothelial cells in direct contact with blood are called vascular endothelial cells, whereas those in direct contact with lymph are known as lymphatic endothelial cells.

Inflammation is associated with a large group of disorders that underlie a variety of diseases. The immune system is often involved with inflammatory disorders, for example allergic reactions and some myopathies, with many immune system disorders resulting in abnormal inflammation. Non-immune diseases with causal origins in inflammatory processes include, without limitation, cancer, atherosclerosis, and ischemic heart disease, among others described herein or identifiable by skilled persons upon reading of the present disclosure. Examples of disorders associated with inflammation include, without limitation, acne vulgaris, atherosclerosis, asthma, autoimmune diseases, autoinflammatory diseases, celiac disease, chronic prostatitis, colitis, diverticulitis, glomerulonephritis, hidradenitis suppurativa, Acquired Immune Deficiency Syndrome, hypersensitivities, inflammatory bowel diseases, interstitial cystitis, ischemia, lichen planus, Mast Cell Activation Syndrome, mastocytosis, otitis, pelvic inflammatory disease, reperfusion injury, rheumatic fever, rheumatoid arthritis, rhinitis, sarcoidosis, transplant rejection, and vasculitis, among others described herein or identifiable by skilled persons upon reading of the present disclosure. Allergic reactions, otherwise known as type 1 hypersensitivity results from an inappropriate immune response, triggering inflammation. A severe allergic inflammatory response may become a systemic response known as anaphylaxis. Inflammatory myopathies cause muscle inflammation and may occur in immune disorders such as systemic sclerosis, dermatomyositis, polymyositis, and inclusion body myositis.

In a normal inflammation response, the acute inflammatory response is typically terminated when no longer needed to prevent unnecessary “bystander” damage to tissues. Failure to terminate acute inflammation may result in chronic inflammation, and may include damage including without limitation cell death in the patient's tissues. When inflammation overwhelms the patient, systemic inflammatory may occur. In such cases, systemic inflammatory response syndrome may be diagnosed. For example, the term sepsis may refer to chronic inflammation associated with an infection. The term bacteremia refers to bacterial sepsis and viremia refers to viral sepsis. Vasodilation and organ dysfunction are serious problems that are often associated with widespread infection and inflammation that may lead to septic shock and death.

Inflammation may involve high systemic levels of acute-phase proteins. In acute inflammation, these proteins may be beneficial; however, in chronic inflammation they can contribute to amyloidosis. These proteins include, without limitation, C-reactive protein, serum amyloid A, and serum amyloid P, among others, which are associated with a range of systemic effects such as fever, increased blood pressure, decreased sweating, malaise, loss of appetite, and somnolence. Inflammation often affects the numbers of leukocytes present in the body of a patient. Leukocytosis often occurs during inflammation induced by infection, where it results in a large increase in the amount of leukocytes in the blood, especially immature cells. In leukocytosis, leukocyte numbers may increase to between, or between about, 15,000 and 20 000 cells per microliter, or up to about, 100 000 cells per microliter. Bacterial infection often results in an increase of neutrophils, creating neutrophilia, whereas diseases such as asthma, hay fever, and parasite infestation may result in an increase in eosinophils, creating eosinophilia.

Systemic inflammation is typically not confined to a particular tissue but may involve the endothelium and other organ systems. Increased levels of several markers of inflammation may be present, such as interleukin-6, Interleukin-8, interleukin-18, Tumor necrosis factor-alpha, and C-reactive protein, among others. Chronic inflammation may last days, months or even years, and may lead to the formation of a chronic wound. Chronic inflammation often involves increased presence of macrophages in the injured tissue, which may release toxins that are injurious to the patient's own tissues.

In some embodiments, the present disclosure relates to compositions and methods for preventing or decreasing inflammation by inhibiting a caspase-9 signaling pathway. Without being limited to a particular mode of action, the caspase-9 acts via a signaling pathway that does not involve modulation of VEGF-A levels, or induction of apoptosis in the cells expressing activated caspase-9.

In some embodiments, the effects may occur in one or more neuronal tissues, including tissues of the central nervous system or the peripheral nervous system such as the brain, spinal cord, nerves, and eye, such as in the retina of the eye. In some embodiments, the effects may occur in one or more other tissues, such as in the gut, blood, lymph, muscle, skeletal, skin, or other tissues described herein or identifiable by skilled persons upon reading the present disclosure.

In some embodiments, the compositions and methods described herein can be used for inhibiting a caspase-9 signaling pathway and thereby decreasing inflammation associated with various pathological conditions. For example, the compositions and methods described herein may be used for reducing retinal inflammation in diseases having an inflammatory component. In some embodiments, the term “neuroinflammation” refers to inflammation of a nervous system tissue. For example, such diseases include without limitation RVO, diabetic macular edema, retinal detachment, ocular trauma, retinitis pigmentosa, age-related macular degeneration (AMD), uveitis, retinal degenerative diseases, and glaucoma, among others described herein or identifiable by skilled persons upon reading the present disclosure. Exemplary retinal diseases include, without limitation, Leber's Hereditary Optic Neuropathy, Leigh Syndrome, Stargadt, retinitis pigmentosa, Best disease, and birdshot retinopathy, among others. The compositions and methods described herein may also be used for reducing ocular inflammation in non-hereditary inflammatory conditions including, without limitation, dry eye disease (conjunctivitis), episcleritis, and atopic dermatitis, among others. In some embodiments, the compositions and methods described herein can be used for treating neuroinflammatory injury in central nervous system (CNS) tissues, such as neuroinflammatory injury in multiple sclerosis, Behcets, Lupus, Systemic sarcoidosis, and central serous chorioretinopathy, among others. In some embodiments, the compositions and methods described herein can be used for treating ocular pathology in systemic neuroinflammatory diseases, such as Behcet's disease, multiple sclerosis, or systemic Lupus erythematosus, among others described herein or identifiable by skilled persons upon reading the present disclosure. In some embodiments, the compositions and methods described herein can be used for treating vasculitis. In some embodiments, the compositions and methods described herein can be used for reducing leukocyte infiltration into tissues, for example to treat sepsis, attenuate cytokine storm, or modulate innate immune response, among others described herein or identifiable by skilled persons upon reading the present disclosure. In some embodiments, the compositions and methods described herein can be used for treating inflammatory conditions including without limitation inflammatory bowel disease (IBD), rhegmatogenous retinal detachment, ischemic stroke, amyotrophic lateral sclerosis (ALS), or atherosclerosis, among others described herein or identifiable by skilled persons upon reading the present disclosure. In some embodiments, the compositions and methods described herein can be used to treat inflammatory consequences of XIAP deficiency disorder or lymphoproliferative syndrome.

Caspases and Diabetic Macular Edema (DME) and/or Retinal Vein Occlusion (RVO)

In some embodiments, the present disclosure relates to compositions and methods for the inhibition of diabetic macular edema (DME) and/or retinal vein occlusion (RVO).

DME is the leading cause of new blindness in the Western world. There are at least 23 million Americans with diabetes mellitus and more than 382 million world-wide; 80% will develop retinopathy and as many as 40% will develop DME. Compounding this problem, only about 50% of patients with diabetes receive proper eye care, and many cases of diabetes are currently not diagnosed. All of this increases the burden of diabetic retinal diseases, including DME and RVO. At present, the only proven pharmacologic option is anti-VEGF therapy which is delivered by intravitreal injection. However, non-compliance is a problem; many patients do not want intraocular injections and miss prescribed doses. Further, it is estimated that as many as 50% of patients with DME will not respond to anti-VEGF therapy. The other treatment is laser photocoagulation, which can reduce vision loss by 50%; the goal is to reduce progression of the disease, however significant improvement of vision is uncommon.

The disclosure of U.S. patent application Ser. No. 16/243,884 provides compositions and method for treating DME and/or RVO in a patient, for example, methods and compositions for inhibiting caspase-9 signaling activity associated with the induction and/or exacerbation of DME and/or RVO in a patient.

As used herein, the term “DME” refers to clinically detectable diabetic macular edema. DME occurs in patients having clinically detectable diabetes mellitus (also referred to herein simply as diabetes), frequently in type 2 diabetes mellitus but also in type 1 diabetes mellitus. Clinical symptoms of DME include retinal edema and diabetic retinopathy with macular edema. DME may be detected using optical coherence tomography (OCT). DME is the major cause of blindness in working age adults (20-70 years old).

As used herein, the term “RVO” refers to clinically detectable retinal vein occlusion. RVO can occur in any patients, but is more common in those also having clinically detectable atherosclerosis, diabetes, hypertension, glaucoma, macular edema, or vitreous hemorrhage. RVO is more common in elderly patients. RVO can cause glaucoma and macular edema, including DME. RVO may be detected using angiography and/or OCT. RVO is the second leading cause of blindness in working age adults.

### Caspase Inhibitors

In some embodiments, the caspase inhibitors of the instant disclosure target one or more of caspases -1, -2, -3, -4, -5, -6, -7, -8, -9, 10, -11, -12, and -14. In certain embodiments, the inhibitor is a non-specific inhibitor of one or more of caspases -1, -2, -3, -4, -5, -6, -7, -8, -9, 10, -11, -12, and -14. In alternative embodiments, the inhibitor is a specific inhibitor of a single caspase or of a particular subset of caspases selected from the group consisting of caspases -1, -2, -3, -4, -5, -6, -7, -8, -9, 10, -11, -12, and -14. In certain embodiments, the specific inhibitor is an inhibitor of caspase-9 or inhibitor of caspase-6.

In certain embodiments, the caspase inhibitors are selected from the group consisting of small molecule inhibitors, nucleic acid inhibitors, and polypeptide inhibitors. Such inhibitors can exert their function by directly or indirectly inhibiting either the expression or activity of a caspase.

In certain embodiments, the caspase inhibitors of the instant disclosure include small molecule inhibitors of caspases. In certain embodiments the small molecule inhibitors of caspases include, but are not limited to, isatin sulfonamides (Lee, et al., J Biol Chem 275:16007-16014 (2000); Nuttall, et al., Drug Discov Today 6:85-91 (2001)), anilinoquinazolines (Scott, et al., JPET 304 (1) 433-440 (2003), and one or more small molecule caspase inhibitor disclosed in U.S. Pat. No. 6,878,743. In certain embodiments, inhibitors of caspases include, without limitation, small molecule inhibitors of apoptotic protease activating factor-1 (Apaf-1), such as compounds Leonurine (also known as SCM-198), ZYZ-488, or QM31 (also known as SVT016426). Leonurine is a natural alkaloid that may occupy the caspase recruitment site of Apaf-1, blocking its interaction with procaspase-9. ZYZ-488 is an inhibitor of Apaf-1 that may inhibit the activation of procaspase-9 and procaspase-3. QM31 may inhibit the formation of the apoptosome, the caspase activation complex composed of Apaf-1, cytochrome c, dATP and caspase-9.

For example, but not by way of limitation, the caspase inhibitors of the instant disclosure which are nucleic acids include, but are not limited to, inhibitors that function by inhibiting the expression of the target, such as ribozymes, antisense oligonucleotide inhibitors, and siRNA inhibitors. A “ribozyme” refers to a nucleic acid capable of cleaving a specific nucleic acid sequence. Within some embodiments, a ribozyme should be understood to refer to RNA molecules that contain anti-sense sequences for specific recognition, and an RNA-cleaving enzymatic activity, see, for example, U.S. Pat. No. 6,770,633. In contrast, “antisense oligonucleotides” generally are small oligonucleotides complementary to a part of a gene to impact expression of that gene. Gene expression can be inhibited through hybridization of an oligonucleotide to a specific gene or messenger RNA (mRNA) thereof. In some cases, a therapeutic strategy can be applied to dampen expression of one or several genes believed to initiate or to accelerate inflammation, see, for example, U.S. Pat. No. 6,822,087 and WO 2006/062716. A “small interfering RNA” or “short interfering RNA” or “siRNA” or “short hairpin RNA” or “shRNA” are forms of RNA interference (RNAi). An interfering RNA can be a double-stranded RNA or partially double-stranded RNA molecule that is complementary to a target nucleic acid sequence, for example, caspase-6 or caspase-9. Micro interfering RNA's (miRNA) also fall in this category. A double-stranded RNA molecule is formed by the complementary pairing between a first RNA portion and a second RNA portion within the molecule. The length of each portion generally is less than 30 nucleotides in length (e.g., 29, 28, 27, 26, 25, 24, 23, 22, 21, 20, 19, 18, 17, 16, 15, 14, 13, 12, 11, or 10 nucleotides). In some embodiments, the length of each portion is 19 to 25 nucleotides in length. In some siRNA molecules, the complementary first and second portions of the RNA molecule are the “stem” of a hairpin structure. The two portions can be joined by a linking sequence, which can form the “loop” in the hairpin structure. The linking sequence can vary in length. In some embodiments, the linking sequence can be 5, 6, 7, 8, 9, 10, 11, 12 or 13 nucleotides in length. Linking sequences can be used to join the first and second portions, and are known in the art. The first and second portions are complementary but may not be completely symmetrical, as the hairpin structure may contain 3′ or 5′ overhang nucleotides (e.g., a 1, 2, 3, 4, or 5 nucleotide overhang). The RNA molecules of the disclosure can be expressed from a vector or produced chemically or synthetically.

In certain embodiments, the caspase inhibitors of the instant disclosure are polypeptide inhibitors of caspases. In certain embodiments the peptide inhibitors of caspases include, but are not limited to Z-LEHD-AMC (SEQ ID NO: 59) (WO 2006056487); z-LEHD-fmk (SEQ ID NO: 60), Z-VAD-FMK, CrmA, and Z-VAD-(2, 6-dichlorobenzoyloxopentanoic acid) (Garcia-Calvo, et al., J. Biol. Chem., 273, 32608-32613 (1998)) among others described herein or identifiable by skilled persons upon reading the present disclosure.

In some embodiments, the caspase inhibitors include, but are not limited to the class of protein inhibitors identified as Inhibitors of Apoptosis (“IAPs”). IAPs generally contain one to three BIR (baculovirus IAP repeats) domains, each consisting of approximately 70 amino acid residues. In addition, certain IAPB also have a RING finger domain, defined by seven cysteines and one histidine (e.g. C3HC4 (SEQ ID NO: 61)) that can coordinate two zinc atoms. Exemplary mammalian IAPB, such as, but not limited to c-IAP1 (Accession No. Q13490.2), cIAP2 (Accession No. Q13489.2), and XIAP (Accession No. P98170.2), each of which have three BIRs in the N-terminal portion of the molecule and a RING finger at the C-terminus. In contrast, NAIP (Accession No. Q13075.3), another exemplary mammalian IAP, contains three BIRs without RING, and survivin (Accession No. 015392.2) and BRUCE (Accession No. Q9H8B7), which are two additional exemplary IAPB, each has just one BIR.

In certain embodiments, the caspase inhibitor is a dominant negative form of a caspase polypeptide. In some embodiments, the dominant negative form of a caspase polypeptide is a dominant negative form of a caspase selected from the group consisting of caspases -1, -2, -3, -4, -5, -6, -7, -8, -9, 10, -11, -12, and -14. For example, but not by way of limitation, the dominant negative form of a caspase polypeptide can be a dominant negative form of caspase-6. In particular embodiments, the dominant negative form of caspase-6 is the polypeptide designated “C6DN” in Denault, J. B. and G. S. Salvesen, Expression, purification, and characterization of caspases. Curr Protoc Protein Sci, 2003. Chapter 21: p. Unit 21 13.

For example, in some embodiments, the dominant negative form of caspase-6 may have the sequence

(SEQ ID NO: 1)
MASSASGLRRGHPAGGEENMTETDAFYKREMFDPAEKYKMDHRRRGIAL
IFNHERFFWHLTLPERRGTCADRDNLTRRFSDLGFEVKCFNDLKAEELL
LKIHEVSTVSHADADCFVCVFLSHGEGNHIYAYDAKIEIQTLTGLFKGD
KCHSLVGKPKIFIIQAARGNQHDVPVIPLDVVDNQTEKLDTNITEVDAA
SVYTLPAGADFLMCYSVAEGYYSHRETVNGSWYIQDLCEMLGKYGSSLE
FTELLTLVNRKVSQRRVDFCKDPSAIGKKQVPCFASMLTKKLHFFPKSN
LEHHHH.

In certain embodiments, the peptide inhibitor of caspase-9 is the third BIR domain of XIAP, referred to herein as “XBIR3”. For example, in some embodiments, the XBIR3 has the sequence

(SEQ ID NO: 2)
STNLPRNPSMADYEARIFTFGTWIYSVNKEQLARAGFYALGEGDKVKCFH
CGGGLTDWKPSEDPWEQHAKWYPGCKYLLEQKGQEYINNIHLTHS.

In certain embodiments, the peptide inhibitor of caspase-9 is XBIR3 having the sequence MGSSHREIHREISSGLVPRGSHMSTNTCLPRNPSMADYEARIFTFGTWIYSVNKEQLARA GFYTDWALGEGDKVKCFHCGGGLRPSEDPWEQHARWYPGCRYLLEQRGQEYINNIHLT HS (SEQ ID NO: 3). The amino acid sequence of SEQ ID NO: 3 includes a His-tag near the N-terminus having the sequence HERRHE (SEQ ID NO: 4). In certain embodiments, the peptide inhibitors of caspase-9 such as that of SEQ ID NO: 3, and others described herein, do not include a His-tag. For example, in some embodiments where the peptide inhibitor of caspase-9 is intended to be administered to a patient, the peptide inhibitor of caspase-9 may lack a His-tag.

In certain embodiments the peptide inhibitor of caspase-9 is XBIR3 having the sequence MGS SHRHIMES SGLVPRGSHMSTNTLPRNP SMADYEARIFTF GTWIYSVNKEQLARAGF YTDWALGEGDKVKCFHCGGGLRP SEDPWEQHARWYPGCRYLLEQRGQEYINNIHLTHS (SEQ ID NO: 5). The amino acid sequence of SEQ ID NO: 5 includes a His-tag near the N-terminus having the sequence HEERHE (SEQ ID NO: 4). In certain embodiments, the peptide inhibitors of caspase-9 such as that of SEQ ID NO: 5, and others described herein, do not include a His-tag. For example, in some embodiments where the peptide inhibitor of caspase-9 is intended to be administered to a patient, the peptide inhibitor of caspase-9 may lack a His-tag.

In certain embodiments, the peptide inhibitor of caspase-7 is the second BIR domain of XIAP, referred to herein as “XBIR2”. For example, in some embodiments, the XBIR2 has the sequence EEARLKSFQNWPDYAHLTPRELASAGLYYTGIGDQVQCFCCGGKLKNWEPCDRAWSE HRRHFPNCFFV (SEQ ID NO: 6), corresponding to residues 163-230 of human XIAP (e.g. Accession No. P98170.2).

In certain embodiments the peptide inhibitor of caspase-7 includes a linker-BIR2, referred to herein as a portion of XIAP comprising the XIAP linker domain and the second BIR domain of XIAP. For example, in certain embodiments, the peptide inhibitor of caspase7 includes a linker-BIR2 comprising residues 124-240 of human XIAP (e.g. Accession No. P98170.2), such as described in Huang et al. Cell 104:781-790, incorporated herein by reference in its entirety. In the paper by Huang et al., it was shown that the direct contact of caspase-7 with XIAP is via the linker domain. In some embodiments, the linker-BIR2 has the sequence RDHFALDRPSETHADYLLRTGQVVDISDTIYPRNPAMYSEEARLKSFQNWPDYAHLTPR ELASAGLYYTGIGDQVQCFCCGGKLKNWEPCDRAWSEHRRHFPNCFFVLGRNLNIRSE (SEQ ID NO. 51), corresponding to residues 163-230 of human XIAP (e.g. Accession No. P98170.2).

Polypeptide caspase inhibitors include those amino acid sequences that retain certain structural and functional features of the identified caspase inhibitor polypeptides, yet differ from the identified inhibitors' amino acid sequences at one or more positions. Such polypeptide variants can be prepared by substituting, deleting, or adding amino acid residues from the original sequences via methods known in the art.

In certain embodiments, such substantially similar sequences include sequences that incorporate conservative amino acid substitutions. As used herein, a “conservative amino acid substitution” is intended to include a substitution in which the amino acid residue is replaced with an amino acid residue having a similar side chain. Families of amino acid residues having similar side chains have been defined in the art, including: basic side chains (e.g., lysine, arginine, histidine); acidic side chains (e.g., aspartic acid, glutamic acid); uncharged polar side chains (e.g., glycine, asparagine, glutamine, serine, threonine, tyrosine, cysteine); nonpolar side chains (e.g., alanine, valine, leucine, isoleucine, proline, phenylalanine, methionine, tryptophan); (3-branched side chains (e.g., threonine, valine, isoleucine); and aromatic side chains (e.g., tyrosine, phenylalanine, tryptophan, histidine). Other generally preferred substitutions involve replacement of an amino acid residue with another residue having a small side chain, such as alanine or glycine. Amino acid substituted peptides can be prepared by standard techniques, such as automated chemical synthesis.

In certain embodiments, a polypeptide caspase inhibitor of the present disclosure has at least, or at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence of a polypeptide caspase inhibitor disclosed herein, and is capable of caspase inhibition. As used herein, the percent identity between two amino acid sequences may be determined using standard software such as BLAST or FASTA. The effect of the amino acid substitutions on the ability of the synthesized polypeptide to inhibit caspases can be tested using the methods disclosed in the Examples sections of U.S. patent application Ser. No. 13/768.687, U.S. Provisional Patent Application No. 62/840,234, and U.S. patent application Ser. No. 16/243,884.

Caspase Inhibitor-Cell Penetrating Peptide Conjugates

In certain embodiments of the instant disclosure, the caspase inhibitor is conjugated to a cell penetrating peptide to form a caspase inhibitor-cell penetrating peptide conjugate, also referred to herein as a cell-penetrating caspase inhibitor conjugate. The caspase inhibitor-cell penetrating peptide conjugate can facilitate delivery of the caspase inhibitor to into a cell.

As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. In certain embodiments, the cell-penetrating peptide used in the membrane-permeable complex of the present disclosure preferably comprises at least one non-functional cysteine residue, which is either free or derivatized to form a disulfide link with the caspase inhibitor, which has been modified for such linkage. Representative amino acid motifs conferring such properties are listed in U.S. Pat. No. 6,348,185, the contents of which are expressly incorporated herein by reference.

Several suitable exemplary cell-penetrating peptides are described by Pescina et al. (2018) Journal of Controlled Release 284:84-102, the disclosure of which is incorporated herein by reference. The cell-penetrating peptides of the present disclosure may include, but are not limited to, Penetratin1, transportan, pIsl, TAT(48-60), pVEC, MTS, MAP, polyarginines, DPV1047, M918, M1073, BPrPr (1-28), MPG, Pep-1, MAP12, MAP17, GALA, p28, PreS2, VT5, Bac 7 [Bac (1-24)], PPR, PRR, SAP, SAP(E), CyLoP-1, gH 625, CPP-C, C105Y, Pep-7, and SG3.

The cell-penetrating peptides of the present disclosure include those sequences that retain certain structural and functional features of the identified cell-penetrating peptides, yet differ from the identified peptides' amino acid sequences at one or more positions. Such polypeptide variants can be prepared by substituting, deleting, or adding amino acid residues from the original sequences via methods known in the art.

In certain embodiments, such substantially similar sequences include sequences that incorporate conservative amino acid substitutions, as described above with regard to caspase inhibitor polypeptides. In certain embodiments, a cell-penetrating peptide of the present disclosure has at least, or at least about, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence of a cell-penetrating peptide disclosed herein and is capable of mediating cell penetration. The effect of the amino acid substitutions on the ability of the synthesized peptide to mediate cell penetration can be tested using the methods disclosed in the Examples sections of U.S. patent application Ser. No. 13/768.687, U.S. Provisional Patent Application No. 62/840,234, and U.S. patent application Ser. No. 16/243,884.

In certain embodiments of the present disclosure, the cell-penetrating peptide of the membrane-permeable complex is Penetratin1, comprising the peptide sequence RQIKIWFQNRRMKWKK (SEQ ID NO: 7), or a conservative variant thereof. As used herein, a “conservative variant” is a peptide having one or more amino acid substitutions, wherein the substitutions do not adversely affect the shape--or, therefore, the biological activity (i.e., transport activity) or membrane toxicity--of the cell-penetrating peptide.

Penetratin1 is a 16-amino-acid polypeptide derived from the third alpha-helix of the homeodomain of Drosophila antennapedia. Its structure and function have been well studied and characterized: Derossi et al., Trends Cell Biol., 8(2):84-87, 1998; Dunican et al., Biopolymers, 60(1):45-60, 2001; Hallbrink et al., Biochim. Biophys. Acta, 1515(2):101-09, 2001; Bolton et al., Eur. J. Neurosci., 12(8):2847-55, 2000; Kilk et al., Bioconjug. Chem., 12(6):911-16, 2001; Bellet-Amalric et al., Biochim. Biophys. Acta, 1467(1):131-43, 2000; Fischer et al., J. Pept. Res., 55(2): 163-72, 2000; Thoren et al., FEBS Lett., 482(3):265-68, 2000.

It has been shown that Penetratin1 efficiently carries avidin, a 63-kDa protein, into human Bowes melanoma cells (Kilk et al., Bioconjug. Chem., 12(6):911-16, 2001). Additionally, it has been shown that the transportation of Penetratin1 and its cargo is non-endocytotic and energy-independent, and does not depend upon receptor molecules or transporter molecules. Furthermore, it is known that penetratin1 is able to cross a pure lipid bilayer (Thoren et al., FEBS Lett., 482(3):265-68, 2000). This feature enables Penetratin1 to transport its cargo, free from the limitation of cell-surface-receptor/-transporter availability. The delivery vector previously has been shown to enter all cell types (Derossi et al., Trends Cell Biol., 8(2):84-87, 1998), and effectively to deliver peptides (Troy et al., Proc. Natl. Acad. Sci. USA, 93:5635-40, 1996), antisense oligonucleotides (Troy et al., J. Neurosci., 16:253-61, 1996; Troy et al., J. Neurosci., 17:1911-18, 1997), siRNA (Davidson et al. J. Neurosci., 24:10040-10046, 2004), or XBIR3 (Akpan et al., J. Neurosci., 31:8894-8904, 2011).

Other non-limiting embodiments of the present disclosure involve the use of the following exemplary cell permeant molecules: RL16 (H-RRLRRLLRRLLRRLRR-OH) (SEQ ID NO: 8), a sequence derived from Penetratin1 with sightly different physical properties (Biochim Biophys Acta. 2008 July-August;1780(7-8):948-59); and RVGRRRRRRRRR (SEQ ID NO: 9), a rabies virus sequence which targets neurons see P. Kumar, H. Wu, J. L. McBride, K. E. Jung, M. H. Kim, B. L. Davidson, S. K. Lee, P. Shankar and N. Manjunath, Transvascular delivery of small interfering RNA to the central nervous system, Nature 448 (2007), pp. 39-43.

Transportan is a 27-amino-acid long peptide containing 12 functional amino acids from the amino terminus of the neuropeptide galanin, and the 14-residue sequence of mastoparan in the carboxyl terminus, connected by a lysine (Pooga et al., FASEB J., 12(1):67-77, 1998). It comprises the amino acid sequence GWTLNSAGYLLGKINLKALAALAKKIL (SEQ ID NO: 10), or a conservative variant thereof.

pIsl is derived from the third helix of the homeodomain of the rat insulin 1 gene enhancer protein (Magzoub et al., Biochim. Biophys. Acta, 1512(1):77-89, 2001; Kilk et al., Bioconjug. Chem., 12(6):911-16, 2001). pIsl comprises the amino acid sequence PVIRVW FQNKRCKDKK (SEQ ID NO: 11), or a conservative variant thereof.

Tat is a transcription activating factor, of 86-102 amino acids, that allows translocation across the plasma membrane of an HIV-infected cell, to transactivate the viral genome (Hallbrink et al., Biochem. Biophys. Acta., 1515(2):101-09, 2001; Suzuki et al., J. Biol. Chem., 277(4):2437-43, 2002; Futaki et al., J. Biol. Chem., 276(8):5836-40, 2001). A small Tat fragment, extending from residues 48-60, has been determined to be responsible for nuclear import (Vives et al., J. Biol. Chem., 272(25):16010-017, 1997); it comprises the amino acid sequence GRKKRRQRRRPPQ (SEQ ID NO: 12), or a conservative variant thereof.

pVEC is an 18-amino-acid-long peptide derived from the murine sequence of the cell-adhesion molecule, vascular endothelial cadherin, extending from amino acid 615-632 (Elmquist et al., Exp. Cell Res., 269(2):237-44, 2001). pVEC comprises the amino acid sequence LLIILRRRIRKQAHAH (SEQ ID NO: 13), or a conservative variant thereof.

MTSs, or membrane translocating sequences, are those portions of certain peptides which are recognized by the acceptor proteins that are responsible for directing nascent translation products into the appropriate cellular organelles for further processing (Lindgren et al., Trends in Pharmacological Sciences, 21(3):99-103, 2000; Brodsky, J. L., Int. Rev. Cyt., 178:277-328, 1998; Zhao et al., J. Immunol. Methods, 254(1-2):137-45, 2001). An MTS of particular relevance is 1VIPS peptide, a chimera of the hydrophobic terminal domain of the viral gp41 protein and the nuclear localization signal from simian virus 40 large antigen; it represents one combination of a nuclear localization signal and a membrane translocation sequence that is internalized independent of temperature, and functions as a carrier for oligonucleotides (Lindgren et al., Trends in Pharmacological Sciences, 21(3):99-103, 2000; Morris et al., Nucleic Acids Res., 25:2730-36, 1997). MPS comprises the amino acid sequence GALFLGWLGAAGSTMGAWSQPKKKRKV (SEQ ID NO: 14), or a conservative variant thereof.

Polyarginines (Rn), such as those containing n=3-12 consecutive arginines (SEQ ID NO: 56), are cationic synthetic polymers of arginine having a flexible, unstructured or random coil structure able to internalize into cells via direct translocation and endocytosis mechanisms (Pescina et al. (2018) Journal of Controlled Release 284:84-102). Polyarginines and conservative variants thereof are suitable cell-penetrating peptides of the present disclosure.

Diatos peptide vector 1047 (DPV1047, Vectocell®, Diatos, France) refers to a polypeptide having the sequence VKRGLKLRHVRPRVTRMDV (SEQ ID NO: 15). DPV1047 is a synthetic cationic polypeptide that allows internalization via an endocytosis mechanism (Pescina et al. (2018) Journal of Controlled Release 284:84-102). DPV1047 or conservative variants thereof are suitable cell-penetrating peptides of the present disclosure.

M918 refers to a polypeptide having the sequence MVTVLFRRLRIRRACGPPRVRV (SEQ ID NO: 16). M918 is a cationic, primary amphipathic polypeptide derived from pl4ARF protein that allows internalization via an endocytosis mechanism (Pescina et al. (2018) Journal of Controlled Release 284:84-102). M918 or conservative variants thereof are suitable cell-penetrating peptides of the present disclosure.

M1073 refers to a polypeptide having the sequence MVRRFLVTLRIRRACGPPRVRV (SEQ ID NO: 17). Like M918, M1073 is a cationic, primary amphipathic polypeptide derived from p14ARF protein that allows internalization via an endocytosis mechanism (Pescina et al. (2018) Journal of Controlled Release 284:84-102). M1073 or conservative variants thereof are suitable cell-penetrating peptides of the present disclosure.

BPrPr (1-28) refers to a polypeptide having the sequence MVKSKIGSWILVLFVAMWSDVGLCKKRP (SEQ ID NO: 18). BPrPr (1-28) is a cationic, primary amphipathic polypeptide derived from bovine prion protein that allows internalization via macropynocitosis (Pescina et al. (2018) Journal of Controlled Release 284:84-102). BPrPr (1-28) or conservative variants thereof are suitable cell-penetrating peptides of the present disclosure.

MPG refers to a polypeptide having the sequence GALFLGFLGAAGSTMGAWSQPKKKRKV (SEQ ID NO: 19). MPG is formed by fusing the nuclear localization sequences (NLSs) of the simian virus 40 (SV40) large T antigen (KKKRKV (SEQ ID NO: 20) to the sequence of the HIV glycoprotein 41 (GALFLGFLGAAGSTMGA (SEQ ID NO: 21)). Pep-1 refers to a polypeptide having the sequence KETWWETWWTEWSQPKKKRKV (SEQ ID NO: 22). Pep-1 is formed by fusing the nuclear localization sequences (NLSs) of the simian virus 40 (SV40) large T antigen (KKKRKV (SEQ ID NO: 20) to a tryptophan-rich cluster (KETWWETWWTEW (SEQ ID NO: 23)). MPG and Pep-1 are both cationic, primary amphipathic polypeptides that allow internalization via endocytosis-independent mechanisms (Pescina et al. (2018) Journal of Controlled Release 284:84-102). MPG, Pep-1 or conservative variants thereof are suitable cell-penetrating peptides of the present disclosure.

Model amphipathic peptides, or MAPs, form a group of peptides that have, as their essential features, helical amphipathicity and a length of at least four complete helical turns (Scheller et al., J. Peptide Science, 5(4):185-94, 1999; Hallbrink et al., Biochim. Biophys. Acta., 1515(2):101-09, 2001). An exemplary MAP comprises the amino acid sequence KLALKLALKALKAALKLA-amide (SEQ ID NO: 24), or a conservative variant thereof. Other exemplary MAPs comprise MAP12, having the sequence LKTLTETLKELTKTLTEL (SEQ ID NO: 25) which is a synthetic anionic, secondary amphipathic, α-helical peptide that allows internalization via an endocytosis-independent mechanism, and MAP17, having the sequence QLALQLALQALQAALQLA (SEQ ID NO: 26), which is an amphipathic, secondary amphipathic, α-helical peptide (Pescina et al. (2018) Journal of Controlled Release 284:84-102). MAP12, MAP17 or conservative variants thereof are suitable cell-penetrating peptides of the present disclosure.

GALA refers to a polypeptide having the sequence WEAALAEALAEALAEHLAEALAEALEALAA (SEQ ID NO: 27). GALA is a synthetic, anionic, secondary amphipathic polypeptide (Pescina et al. (2018) Journal of Controlled Release 284:84-102). GALA or conservative variants thereof are suitable cell-penetrating peptides of the present disclosure.

p28 refers to a polypeptide having the sequence LSTAADMQGVVTDGMASGLDKDYLKPDD (SEQ ID NO: 28). p28 is an anionic, secondary amphipathic peptide derived from the bacterial protein azurin. p28 allows internalization via a caveolae-mediated mechanism (Pescina et al. (2018) Journal of Controlled Release 284:84-102). p28 or conservative variants thereof are suitable cell-penetrating peptides of the present disclosure.

PreS2 refers to an amphipathic peptide having the sequence PLSSIFSRIGDP (SEQ ID NO: 29) derived from the PreS2-domain of hepatitis-B virus surface antigens (Pescina et al. (2018) Journal of Controlled Release 284:84-102). PreS2 or conservative variants thereof are suitable cell-penetrating peptides of the present disclosure.

VT5 refers to a synthetic, secondary amphipathic peptide having the sequence DPKGDPKGVTVTVTVTVTGKGDPKPD (SEQ ID NO: 30) and comprising β-sheet structure (Pescina et al. (2018) Journal of Controlled Release 284:84-102). VT5 or conservative variants thereof are suitable cell-penetrating peptides of the present disclosure.

Bac (1-24) refers to a polypeptide having the sequence RRIRPRPPRLPRPRPRPLPFPRPG (SEQ ID NO: 31), comprising residues 1-24 of bactenecin-7 (Bac7), a 59-residue antimicrobial protein. Bac (1-24) is a cationic, polyproline II helical polypeptide that allows internalization via endocytosis mechanisms (Pescina et al. (2018) Journal of Controlled Release 284:84-102). Bac (1-24) or conservative variants thereof are suitable cell-penetrating peptides of the present disclosure.

PPR and PRR refer to synthetic, cationic, proline-rich polypeptides having the sequence (PPR)ₙ (SEQ ID NO: 57) and (PRR)ₙ (SEQ ID NO: 58), respectively, wherein n=3-6, and having a polyproline II helical structure (Pescina et al. (2018) Journal of Controlled Release 284:84-102). PPR, PRR or conservative variants thereof are suitable cell-penetrating peptides of the present disclosure.

SAP refers to an amphipathic, polyproline II helical polypeptide based on a γ-zein sequence. SAP has the sequence VRLPPPVRLPPPVRLPPP (SEQ ID NO: 32). SAP(E) refers to a synthetic, anionic, polyproline II helical polypeptide variant of SAP having the sequence VELPPPVELPPPVELPPP (SEQ ID NO: 33). SAP and SAP(E) allow internalization via an endocytosis mechanism (Pescina et al. (2018) Journal of Controlled Release 284:84-102). SAP, SAP(E) or conservative variants thereof are suitable cell-penetrating peptides of the present disclosure.

CyLoP-1 refers to an amphipathic polypeptide having the sequence CRWRWKCCKK (SEQ ID NO: 34) derived from the nuclear localization domain crot (27-39) of the snake venom toxin crotamine (Pescina et al. (2018) Journal of Controlled Release 284:84-102). CyLoP-1 or conservative variants thereof are suitable cell-penetrating peptides of the present disclosure.

gH 625 refers to a hydrophobic, α-helical polypeptide having the sequence HGLASTLTRWAHYNALIRAF (SEQ ID NO: 35), derived from Herpes simplex virus type I (Pescina et al. (2018) Journal of Controlled Release 284:84-102). gH 625 or conservative variants thereof are suitable cell-penetrating peptides of the present disclosure.

CPP-C refers to a hydrophobic polypeptide having the sequence PIEVCMYREP (SEQ ID NO: 36) derived from the C-terminal region (residues 140-149) of the fibroblast-growth factor 12 (FGF12) (Pescina et al. (2018) Journal of Controlled Release 284:84-102). CPP-C or conservative variants thereof are suitable cell-penetrating peptides of the present disclosure.

C105Y refers to a synthetic hydrophobic polypeptide having the sequence CSIPPEVKFNKPFVYLI (SEQ ID NO: 37) derived from amino acid sequences corresponding to residues 359-374 and a C-terminal domain of alpha-1-antitrypsin (Pescina et al. (2018) Journal of Controlled Release 284:84-102). C105Y or conservative variants thereof are suitable cell-penetrating peptides of the present disclosure.

Pep-7 and SG3 refer to hydrophobic polypeptides obtained from randomized peptide libraries using phage display- or plasmid display-based functional selection platforms, respectively (Pescina et al. (2018) Journal of Controlled Release 284:84-102). Pep-7 has the sequence SDLWEMMMVSLACQY (SEQ ID NO: 38) and SG3 has the sequence RLSGMNEVLSFRW (SEQ ID NO: 39). Pep-7, SG3 or conservative variants thereof are suitable cell-penetrating peptides of the present disclosure.

In certain embodiments, the cell-penetrating peptides and the caspase inhibitors described above are covalently bound to form caspase inhibitor-cell penetrating peptide conjugates. In certain embodiments the cell-penetrating peptide is operably linked to a caspase inhibitor via recombinant DNA technology. For example, in embodiments where the caspase inhibitor is a peptide or polypeptide sequence, a nucleic acid sequence encoding that caspase inhibitor can be introduced either upstream (for linkage to the amino terminus of the cell-penetrating peptide) or downstream (for linkage to the carboxy terminus of the cell-penetrating peptide), or both, of a nucleic acid sequence encoding the caspase inhibitor of interest. Such fusion sequences comprising both the caspase inhibitor encoding nucleic acid sequence and the cell-penetrating peptide encoding nucleic acid sequence can be expressed using techniques well known in the art.

In certain embodiments the caspase inhibitor can be operably linked to the cell-penetrating peptide via a non-covalent linkage. In certain embodiments such non-covalent linkage is mediated by ionic interactions, hydrophobic interactions, hydrogen bonds, or van der Waals forces.

In certain embodiments the caspase inhibitor is operably linked to the cell penetrating peptide via a chemical linker. Examples of such linkages typically incorporate 1-30 nonhydrogen atoms selected from the group consisting of C, N, O, S and P. Exemplary linkers include, but are not limited to, a substituted alkyl or a substituted cycloalkyl. Alternately, the heterologous moiety may be directly attached (where the linker is a single bond) to the amino or carboxy terminus of the cell-penetrating peptide. When the linker is not a single covalent bond, the linker may be any combination of stable chemical bonds, optionally including, single, double, triple or aromatic carbon-carbon bonds, as well as carbon-nitrogen bonds, nitrogen-nitrogen bonds, carbon-oxygen bonds, sulfur-sulfur bonds, carbon-sulfur bonds, phosphorus-oxygen bonds, phosphorus-nitrogen bonds, and nitrogen-platinum bonds. In certain embodiments, the linker incorporates less than 20 nonhydrogen atoms and are composed of any combination of ether, thioether, urea, thiourea, amine, ester, carboxamide, sulfonamide, hydrazide bonds and aromatic or heteroaromatic bonds. In certain embodiments, the linker is a combination of single carbon-carbon bonds and carboxamide, sulfonamide or thioether bonds.

A general strategy for conjugation involves preparing the cell-penetrating peptide and the caspase inhibitor components separately, wherein each is modified or derivatized with appropriate reactive groups to allow for linkage between the two. The modified caspase inhibitor is then incubated together with a cell-penetrating peptide that is prepared for linkage, for a sufficient time (and under such appropriate conditions of temperature, pH, molar ratio, etc.) as to generate a covalent bond between the cell-penetrating peptide and the apoptotic target inhibitor molecule.

Numerous methods and strategies of conjugation will be readily apparent to one of ordinary skill in the art. By way of example only, one such strategy for conjugation is described below, although other techniques, such as the production of fusion proteins or the use of chemical linkers is within the scope of the instant disclosure.

In certain embodiments, when generating a disulfide bond between the caspase inhibitor molecule and the cell-penetrating peptide of the present disclosure, the caspase inhibitor molecule can be modified to contain a thiol group, and a nitropyridyl leaving group can be manufactured on a cysteine residue of the cell-penetrating peptide. Any suitable bond (e.g., thioester bonds, thioether bonds, carbamate bonds, etc.) can be created according to methods generally and well known in the art. Both the derivatized or modified cell-penetrating peptide, and the thiol-containing caspase inhibitor are reconstituted in RNase/DNase sterile water, and then added to each other in amounts appropriate for conjugation (e.g., equimolar amounts). The conjugation mixture is then incubated, for example, for 15 min at 65° C., followed by 60 min at 37° C., and then stored at 4° C. Linkage can be checked, for example, by running the cell-penetrating peptide-linked caspase inhibitor molecule on a 15% non-denaturing PAGE. Apoptotic target inhibitor molecules can then be visualized with the appropriate stain.

In certain embodiments the caspase inhibitor-cell penetrating peptide conjugate will comprise a double stranded nucleic acid conjugated to a cell-penetrating peptide. In the practice of certain of such embodiments, at least one strand of the double-stranded ribonucleic acid molecule (either the sense or the antisense strand) may be modified for linkage with a cell-penetrating peptide (e.g., with a thiol group), so that the covalent bond links the modified strand to the cell-penetrating peptide. Where the strand is modified with a thiol group, the covalent bond linking the cell-penetrating peptide and the modified strand of the ribonucleic acid molecule can be a disulfide bond, as is the case where the cell-penetrating peptide has a free thiol function (i.e., pyridyl disulfide or a free cysteine residue) for coupling. However, it will be apparent to those skilled in the art that a wide variety of functional groups may be used in the modification of the ribonucleic acid, so that a wide variety of covalent bonds (e.g., ester bonds, carbamate bonds, sulfonate bonds, etc.) may be applicable. Additionally, the membrane-permeable complex of the present disclosure may further comprise a moiety conferring target-cell specificity to the complex.

In certain embodiments, the present disclosure is directed to a Penetratin1- XBIR3 (Pen1-XBIR3) conjugate in which the caspase-9 inhibitor and the cell-penetrating peptide are linked by a disulfide bond. In certain of such embodiments, the sequence of the Pen-1-XBIR3 is:

C(NPys)-RQIKIWFQNRRMKWKK-s-s-
MGSSHHHHHHSSGLVPRGSHMSTNTCLPRNPSMADYEARIFTFGTWIYSV
NKEQLARAGFYTDWALGEGDKVKCFHCGGGLRPSEDPWEQHARWYPGCRY
LLEQRGQEYINNIHLTHS
(SEQ ID NOS 40 and 3, respectively, in order of
appearance).

In other of such embodiments, the sequence of the Pen1-XBIR3 is:

C(NPys)-RQIKIWFQNRRMKWKK-s-s-
MGSSHHHHHHSSGLVPRGSHMSTNTLPRNPSMADYEARIFTFGTWIYSVN
KEQLARAGFYTDWALGEGDKVKCFHCGGGLRPSEDPWEQHARWYPGCRYL
LEQRGQEYINNIHLTHS
(SEQ ID NOS 40 and 5, respectively, in order of
appearance).

In other of such embodiments, the His-tag comprised in SEQ ID NO: 3 and SEQ ID NO: 5 can be removed such that the respective Pen-1-XBIR3 lacks a His-tag.

In some embodiments, the sequence of the Pen1-XBIR3 is:

C(NPys)-RQIKIWFQNRRMKWKK-s-s-MSTNLPRNPSMADYEARIFTF
GTWIYSVNKEQLARAGFYALGEGDKVKCFHCGGGLTDWKPSEDPWEQHAK
WYPGCKYLLEQKGQEYINNIHLTHS
(SEQ ID NOS 40 and 53, respectively, in order of
appearance).

In certain embodiments, the present disclosure is directed to a Penetratin1-XBIR2 (Pen1-XBIR2) conjugate in which the caspase-7 inhibitor and the cell-penetrating peptide are linked by a disulfide bond. In certain of such embodiments, the sequence of the Pen-1-XBIR2 is:

C(NPys)-RQIKIWFQNRRMKWKK-s-s-MEEARLKSFQNWPDYAHLTPR
ELASAGLYYTGIGDQVQCFCCGGKLKNWEPCDRAWSEHRRHFPNCFFV
(SEQ ID NOS 40 and 54, respectively, in order of
appearance).

In certain embodiments, the present disclosure is directed to a Penetratin1-linker-BIR2 (Pen1-linker-BIR2) conjugate in which the caspase-7 inhibitor and the cell-penetrating peptide are linked by a disulfide bond. In certain of such embodiments, the sequence of the Pen-1-linker-BIR2 is:

C(NPys)-RQIKIWFQNRRMKWKK-s-s-
MRDHFALDRPSETHADYLLRTGQVVDISDTIYPRNPAMYSEEARLKSFQN
WPDYAHLTPRELASAGLYYTGIGDQVQCFCCGGKLKNWEPCDRAWSEHRR
HFPNCFFVLGRNLNIRSE
(SEQ ID NOS 40 and 55, respectively, in order of
appearance).

In certain embodiments, the present disclosure is directed to a conjugate of Penetratinl and a dominant negative form of a caspase polypeptide. In certain of such embodiments, the dominant negative form of caspase-6 is the polypeptide designated “C6DN” in Denault, J. B. and G. S. Salvesen, Expression, purification, and characterization of caspases. Curr Protoc Protein Sci, 2003. Chapter 21: p. Unit 21 13, and the sequence of penetratin1-C6DN is

RQIKIWFQNRRMKWKK-s-s-
MASSASGLRRGHPAGGEENMTETDAFYKREMFDPAEKYKMDHRRRGIALI
FNHERFFWHLTLPERRGTCADRDNLTRRFSDLGFEVKCFNDLKAEELLLK
IHEVSTVSHADADCFVCVFLSHGEGNHIYAYDAKIEIQTLTGLFKGDKCH
SLVGKPKIFIIQAARGNQHDVPVIPLDVVDNQTEKLDTNITEVDAASVYT
LPAGADFLMCYSVAEGYYSHRETVNGSWYIQDLCEMLGKYGSSLEFTELL
TLVNRKVSQRRVDFCKDPSAIGKKQVPCFASMLTKKLHFFPKSNLEHHHH
(SEQ ID NOS 7 and 1, respectively, in order of
appearance).

### High Concentration Caspase Inhibitor-Cell Penetrating Peptide Conjugates

As discussed above, caspase inhibitor-cell penetrating peptide conjugates have previously been shown to be efficacious in cell culture and rat and mouse studies to inhibit apoptosis associated with ischemic injury in the CNS (U.S. patent application Ser. No. 13/768.687, filed on Feb. 15, 2013, and published as U.S. Patent Application Publication No. US 2014/0024597), or inhibition of diabetic macular edema (DME) and/or retinal vein occlusion (RVO) (U.S. patent application Ser. No. 016/243,884, filed on Jan. 9, 2019, and published as U.S. Patent Application Publication No. US 2019/0142915), and preventing or reducing inflammation (U.S. Provisional Patent Application No. 62/840,234, filed on Apr. 29, 2019).

Administration of pharmaceutical compositions in larger animals and humans typically requires production of pharmaceutical compositions in higher concentrations for improved efficacy. However, production of high concentration compositions of cell-penetrating forms of caspase inhibitors has proved challenging.

In some embodiments, the present disclosure provides high concentration caspase inhibitor-cell penetrating peptide conjugates that allow improved efficacy for inhibiting apoptosis associated with ischemic injury in the CNS or apoptosis associated with neurodegenerative disorders, or inhibition of DME and/or retinal vein occlusion RVO, or preventing or reducing inflammation when administered to a patient.

In some embodiments, the term “high concentration” as used herein refers to a concentration greater than 0.5 mM, such as a concentration of, or of about, 0.5 mM to 100 mM. Accordingly, in some embodiments, the present disclosure provides compositions of caspase inhibitor-cell penetrating peptide conjugates having a concentration greater than 0.5 mM, for example having a concentration of, or of about, 0.5 mM to 100 mM. For example, in some embodiments, the present disclosure provides a caspase inhibitor-cell penetrating peptide conjugate at a concentration of, or of about, 0.5 mM to 10 mM, such as a concentration of, or of about, 0.5 mM, 1 mM, 2 mM, 3 mM, 4 mM, 5 mM, 6 mM, 7 mM, 8 mM, 9 mM, or 10 mM. In particular, such high concentration caspase inhibitor-cell penetrating peptide conjugate described herein may be suitable in some embodiments for administration to a human patient, and are expected to provide improved efficacy of treatment in a human patient when compared to the same caspase inhibitor-cell penetrating peptide conjugate administered at a lower concentration. For example, a 1% dosing for a Pen1-XBIR3 equates to approximately 0.7 mM in a human patient.

In certain embodiments, the present disclosure provides high concentration compositions of disulfide linked caspase inhibitor-cell penetrating peptide conjugates.

In some embodiments, the high concentration disulfide linked caspase inhibitor-cell penetrating peptide conjugate may comprise any caspase inhibitor of the present disclosure linked to any cell-penetrating peptide of the present disclosure. For example, the caspase inhibitor may be selected from small molecule inhibitors, nucleic acid inhibitors, and peptide/protein inhibitors. For example, in some embodiments, the high concentration disulfide linked caspase inhibitor-cell penetrating peptide conjugate may be selected from XBIR3, XBIR2, and a dominant-negative caspase, such as a dominant negative caspase -1, -2, -3, -4, -5, -6, -7, -8, -9, 10, -11, -12, or -14. For example, in some embodiments, the cell-penetrating peptide that is disulfide linked to the caspase inhibitor may be selected from Penetratin1, transportan, pIsl, TAT(48-60), pVEC, MTS, MAP, polyarginines, DPV1047, M918, M1073, BPrPr (1-28), MPG, Pep-1, MAP12, MAP17, GALA, p28, PreS2, VT5, Bac 7 [Bac (1-24)], PPR, PRR, SAP, SAP(E), CyLoP-1, gH 625, CPP-C, C105Y, Pep-7, and SG3.

In some embodiments, the present disclosure describes methods of providing high concentration disulfide linked caspase inhibitor-cell penetrating peptide conjugates. For example, when the caspase inhibitor comprises one or more thiol group, such as comprised in one or more cysteine residues, the thiol groups are susceptible to oxidation resulting in the disulfide derivative cystine, which can cause dimerization of the caspase inhibitor, and which may prevent efficient linkage of the caspase inhibitor to a cell-penetrating peptide, preventing production of a disulfide linked disulfide linked caspase inhibitor-cell penetrating peptide conjugate in high concentration. Accordingly, the present disclosure provides a solution to this technical problem, wherein the caspase inhibitor is treated with a reducing agent to provide a reduced caspase inhibitor that has decreased dimer formation compared to a caspase inhibitor that has not been treated with the reducing agent. The reduced caspase inhibitor is then incubated with the cell-penetrating peptide to provide a high concentration disulfide linked caspase inhibitor-cell penetrating peptide conjugates as described herein.

In some embodiments, the caspase inhibitor treated with the reducing agent has no more than 40% caspase inhibitor dimers. Typically, in some embodiments, the caspase inhibitor treated with the reducing agent has no more than 10% caspase inhibitor dimers.

In some embodiments, the caspase inhibitor treated with the reducing agent has 70 to 99% decrease in dimer formation that the caspase inhibitor that has not been treated with the reducing agent. In some embodiments, the caspase inhibitor treated with the reducing agent has at least 3-fold decrease in dimer formation that the caspase inhibitor that has not been treated with the reducing agent, such as 3-fold to 4-fold decrease in dimer formation that the caspase inhibitor that has not been treated with the reducing agent.

In some embodiments, the caspase inhibitor treated with the reducing agent has increased solubility compared with the caspase inhibitor that has not been treated with the reducing agent. For example, without treatment of the XBIR3 with a reducing agent, there is typically precipitation of Pen1-XBIR3 at concentrations of 50 μM or greater. In contrast, in some embodiments, treatment of XBIR3 with a reducing agent allows linkage of the reduced XBIR3 with a cell penetrating peptide at high concentration without precipitation. For example, in some embodiments, treatment of XBIR3 with a reducing agent allows linkage of the reduced XBIR3 with a cell penetrating peptide at concentrations of, or of about, 0.5 mM, 1 mM, 2 mM, 3 mM, 4 mM, 5 mM, 6 mM, 7 mM, 8 mM, 9 mM, or 10 mM, without precipitation. In some embodiments, treatment of XBIR3 with a reducing agent allows linkage of the reduced XBIR3 with a cell penetrating peptide at concentrations of, or of about, 0.5 mM, 1 mM, 2 mM, 3 mM, 4 mM, 5 mM, 6 mM, 7 mM, 8 mM, 9 mM, or 10 mM, without substantial precipitation. In some embodiments, treatment of XBIR3 with a reducing agent allows linkage of the reduced XBIR3 with a cell penetrating peptide at concentrations of, or of about, 0.5 mM, 1 mM, 2 mM, 3 mM, 4 mM, 5 mM, 6 mM, 7 mM, 8 mM, 9 mM, or 10 mM, with decreased precipitation, such as, for example, at least, or at least about, 10%, 20%, 30%, 40%, 50%, 60%, 70%, 80%, or 90% less precipitation than without treatment of the XBIR3 with a reducing agent. In some embodiments, the decreased precipitation may occur at the time of linkage. In other embodiments, the decreased precipitation may occur following linkage, such as during storage of the XBIR3 linked with a cell penetrating peptide, over a period of storage time that may be up to an hour, a day, a week, a month, a year, or several years.

In some embodiments, the reducing agent is dithiothreitol (DTT). As would be understood by skilled persons, DTT is the common name for a small-molecule redox reagent also known as Cleland's reagent. DTT is a reducing agent; once oxidized, it forms a stable six-membered ring with an internal disulfide bond. Generally, DTT is used as a protecting agent that prevents oxidation of thiol groups. DTT may be used as a reducing or “deprotecting” agent for thiolated nucleoic acids. Accordingly, in some embodiments, DTT may be used as an agent for reducing nucleic acid caspase inhibitors of the present disclosure. DTT may also be used to reduce the disulfide bonds of proteins and, more generally, to prevent intramolecular and intermolecular disulfide bonds from forming between cysteine residues of proteins. Accordingly, in some embodiments, DTT may be used as an agent for reducing polypeptide caspase inhibitors of the present disclosure.

In some embodiments, the reducing agent is 2-mercaptoethanol (also known as β-mercaptoethanol, BME, 2BME, 2-ME or β-met). As would be understood by skilled persons, 2-ME is commonly used to reduce disulfide bonds. Polypeptide caspase inhibitors of the present disclosure can be denatured by 2-ME, which cleaves the disulfide bonds that may form between thiol groups of cysteine residues.

In some embodiments, the reducing agent is TCEP (tris(2-carboxyethyl)phosphine). As would be understood by skilled persons, TCEP is non-volatile, odorless, and unlike most other reducing agents, is resistant to air oxidation. Compared to DTT, TCEP is more stable, more effective, and able to reduce disulfide bonds at lower pHs. TCEP effectively reduces disulfide bonds over a broad pH range. Polypeptide caspase inhibitors of the present disclosure can be denatured by TCEP.

Following the reducing step, and prior to disulfide linkage of the caspase inhibitor with a cell-penetrating peptide, the reducing agent can be removed from the reduced caspase inhibitor. For example, the reducing agent can be removed from the caspase inhibitor using any suitable method, such as suitable ultrafiltration method, electrophoresis purification method, or dialysis purification method identifiable by skilled persons upon reading the present disclosure. Following removal of the reducing agent from the reduced caspase inhibitor, the reducing agent may comprise 0.001% or less of a composition comprising the caspase inhibitor. The purification steps can include a step of buffer exchange, to provide a reduced caspase inhibitor comprised in a composition that includes a pharmaceutically acceptable carrier.

For example, Example 1 describes a method of reducing an exemplary caspase inhibitor of the present disclosure, XBIR3, using DTT, followed by spin column filtration and buffer exchange into phosphate buffered saline (PBS) and further remove the DTT from the reduced XBIR3

Filtration methods suitable for purification of the reduced caspase inhibitor may include any identifiable suitable diafiltration, or ultrafiltration methods, that can be used for buffer exchange through the use of centrifugal force or other external pressure, such as mechanical pump pressure or gas pressure, to drive small microsolutes such as the reducing agent through a porous membrane, while preventing macrosolutes (larger than the pore size), such as the reduced caspase inhibitor, to pass through. One advantage of using such filtration methods is that the reducing agent can be rapidly removed from the reduced caspase inhibitor prior to disulfide linkage with the cell penetrating peptide.

Suitable electrophoresis purification methods identifiable by skilled persons upon reading the present disclosure may be used to remove the reducing agent from the reduced caspase inhibitor, provided that the separation can be performed rapidly enough and under conditions to prevent substantial re-oxidation of the thiol groups of the caspase inhibitor. For example, electrophoretic microfluidic methods may be used to rapidly separate caspase inhibitors from reducing agents.

Dialysis refers to a method for desalting (removing microsolutes) or buffer/solvent exchange, using osmotic pressure to drive solutes across a membrane. Any dialysis method identifiable by skilled persons may be used to remove the reducing agent and buffer exchange provided that the dialysis can be performed rapidly enough such that re-oxidation of the thiol groups of the caspase inhibitor is kept to a minimum.

In general, purification of the reduced caspase inhibitor, including removal of excess reducing agent and buffer exchange, is preferably perfomed rapidly to prevent re-oxidation of the thiol groups of the caspase inhibitor before disulfide linkage of the reduced caspase inhibitor with the cell penetrating peptide. For example, the purification step preferably is performed rapidly such that there is less than 30 minutes between completion of the reducing step and the beginning of the step of disulfide linkage of the reduced caspase inhibitor to the cell penetrating peptide.

Rapidly following purification of the reduced caspase inhibitor, the reduced caspase inhibitor is incubated with a cell penetrating peptide to provide a disulfide linked caspase inhibitor-cell penetrating peptide conjugate, as described herein.

Accordingly, in some embodiments, the present disclosure provides methods of providing high concentration disulfide linked caspase inhibitor-cell penetrating peptide conjugates. The method includes, without limitation, incubating a caspase inhibitor having one or more thiol groups with a reducing agent selected from dithiothreitol (DTT) and 2-mercaptoethanol (2-ME) to provide a reduced caspase inhibitor; removing the reducing agent from the reduced caspase inhibitor; and conjugating the reduced caspase inhibitor with a cell-penetrating peptide by a disulfide linkage. In some embodiments, the caspase inhibitor may be selected from the group consisting of a caspase -1, -2, -3, -4, -5, -6, -7, -8, -9, -10, -11, -12, and -14 inhibitor. In some embodiments, the caspase inhibitor may be selected from a XBIR3, a XBIR2, a linker-BIR2, and a dominant-negative caspase 6. In some embodiments, the step of removing may be reducing agent may be by filtration. In some embodiments, the method may further include, without limitation, one or more buffer exchange steps, wherein following the buffer exchange step, the reduced caspase inhibitor is comprised in a pharmaceutically acceptable excipient. Accordingly, in some embodiments, the present disclosure provides a composition comprising a disulfide linked caspase inhibitor-cell penetrating peptide conjugate and a pharmaceutically acceptable carrier, wherein the caspase inhibitor-cell penetrating peptide conjugate has a concentration greater than 1 mM. For example, in some embodiments, the the caspase inhibitor-cell penetrating peptide conjugate may have a concentration of up to 1.1. 1.2, 1.3, 1.4, 1.5, 1.6, 1.7, 1.8, 1.9, 2, 3, 4, 5, 6, 7, 8, 9, 10, 20, 30, 40, 50, 60, 70, 80, 90, or 100 mM.

In some embodiments, linkage of the caspase inhibitor to the cell penetrating peptide using the methods described herein does not substantially alter the secondary structure of the caspase inhibitor. Secondary structure of the caspase inhibitors and caspase inhibitor-cell penetrating peptide conjugates described herein can be analyzed by methods identifiable by persons of ordinary skill in the art upon reading the present disclosure. For example, methods such as circular dichroism spectral analysis can be used to analyze protein secondary structure of the caspase inhibitors and caspase inhibitor-cell penetrating peptide conjugates described herein. For example, as shown in Example 4, circular dichroism results show that the secondary structure of both XBIR3 and Pen1-XBIR3 is composed of approximately 10% helix and approximately 35% anti-parallel β-sheet. Thus, linkage of XBIR3 to Penl did not markedly change the XBIR3 secondary structure. Furthermore, the secondary structure was not markedly changed by changes in temperature or concentration. For example, in some embodiments, linkage of the caspase inhibitor to the cell penetrating peptide using the methods described herein may not change the percentage of protein secondary structural content of helices, anti-parallel β-sheets, or both, of the caspase inhibitor by more than, or by more than about, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, or 50%.

### Pharmaceutical Compositions

In some embodiments, the caspase inhibitors or caspase inhibitor-cell penetrating peptide conjugates of the instant disclosure are formulated for administration via injection, inhalation, or topical administration. Injection may be intravenous, intraocular, intraarterial, intracerebral, intracerebroventricular, or sub-tenon's injection. Topical administration may be via eye drops, or directly onto skin, or intranasally.

For intranasal administration, solutions or suspensions comprising the caspase inhibitors or caspase inhibitor-cell penetrating peptide conjugates of the instant disclosure can be formulated for direct application to the nasal cavity by conventional means, for example with a dropper, pipette or spray. Other means for delivering the nasal spray composition, such as inhalation via a metered dose inhaler (MDI), may also be used according to the present disclosure. Several types of MDIs are regularly used for administration by inhalation. These types of devices can include breath-actuated MDI, dry powder inhaler (DPI), spacer/holding chambers in combination with MDI, and nebulizers. The term “MDI” as used herein refers to an inhalation delivery system comprising, for example, a canister containing an active agent dissolved or suspended in a propellant optionally with one or more excipients, a metered dose valve, an actuator, and a mouthpiece. The canister is usually filled with a solution or suspension of an active agent, such as the nasal spray composition, and a propellant, such as one or more hydrofluoroalkanes. When the actuator is depressed a metered dose of the solution is aerosolized for inhalation. Particles comprising the active agent are propelled toward the mouthpiece where they may then be inhaled by a subject. The formulations may be provided in single or multidose form. For example, in the case of a dropper or pipette, this may be achieved by the patient administering an appropriate, predetermined volume of the solution or suspension. In the case of a spray, this may be achieved for example by means of a metering atomising spray pump. To improve nasal delivery and retention the components according to the disclosure may be encapsulated with cyclodextrins, or formulated with agents expected to enhance delivery and retention in the nasal mucosa.

Commercially available administration devices that are used or can be adapted for intranasal administration of a composition of the disclosure include the AERONEB™ (Aerogen, San Francisco, Calif.), AERONEB GO™ (Aerogen); PARI LC PLUS™, PARI BOY™ N, PARI™ eflow (a nebulizer disclosed in U.S. Pat. No. 6,962,151), PARI LC SINUS™, PARI SINUSTAR™, PARI SINUNEB™, VibrENT™ and PARI DURANEB™ (PART Respiratory Equipment, Inc., Monterey, Calif. or Munich, Germany); MICROAIR™ (Omron Healthcare, Inc, Vernon Hills, Ill.), HALOLITE™ (Profile Therapeutics Inc, Boston, Mass.), RESPIMAT™ (Boehringer Ingelheim, Germany), AERODOSE™ (Aerogen, Inc, Mountain View, Calif.), OMRON ELITE™ (Omron Healthcare, Inc, Vernon Hills, Ill.), OMRON MICROAIR™ (Omron Healthcare, Inc, Vernon Hills, Ill.), MABISMIST™ II (Mabis Healthcare, Inc, Lake Forest, Ill.), LUMISCOPE™ 6610, (The Lumiscope Company, Inc, East Brunswick, N.J.), AIRSEP MYSTIQUE™, (AirSep Corporation, Buffalo, N.Y.), ACORN-1™ and ACORN-II™ (Vital Signs, Inc, Totowa, N.J.), AQUATOWER™ (Medical Industries America, Adel, Iowa), AVA-NEB™ (Hudson Respiratory Care Incorporated, Temecula, Calif.), AEROCURRENT™ utilizing the AEROCELL™ disposable cartridge (AerovectRx Corporation, Atlanta, Ga.), CIRRUS™ (Intersurgical Incorporated, Liverpool, N.Y.), DART™ (Professional Medical Products, Greenwood, S.C.), DEVILBISS™ PULMO AIDE (DeVilbiss Corp; Somerset, Pa.), DOWNDRAFT™ (Marquest, Englewood, Colo.), FAN JET™ (Marquest, Englewood, Colo.), mb-5™ (Mefar, Bovezzo, Italy), MISTY NEB™ (Baxter, Valencia, Calif.), SALTER 8900™ (Salter Labs, Arvin, Calif.), SIDESTREAM™ (Medic-Aid, Sussex, UK), UPDRAFT-II™ (Hudson Respiratory Care; Temecula, Calif.), WHISPER JET™ (Marquest Medical Products, Englewood, Colo.), AIOLOS™ (Aiolos Medicnnsk Teknik, Karlstad, Sweden), INSPIRON™ (Intertech Resources, Inc., Bannockburn, Ill.), OPTIMIST™ (Unomedical Inc., McAllen, Tex.), PRODOMO™, SPIRA™ (Respiratory Care Center, Hameenlinna, Finland), AERx™ Essence™ and Ultra™, (Aradigm Corporation, Hayward, Calif.), SONIK™ LDI Nebulizer (Evit Labs, Sacramento, Calif.), ACCUSPRAY™ (BD Medical, Franklin Lake, N.J.), ViaNase ID™ (electronic atomizer; Kurve, Bothell, Wash.), OptiMist™ device or OPTINOSE™ (Oslo, Norway), MAD Nasal™ (Wolfe Tory Medical, Inc., Salt Lake City, Utah), Freepod™ (Valois, Marly le Roi, France), Dolphin™ (Valois), Monopowder™ (Valois), Equadel™ (Valois), VP3™ and VP7™ (Valois), VP6 Pump™ (Valois), Standard Systems Pumps™ (Ing. Erich Pfeiffer, Radolfzell, Germany), AmPump™ (Ing. Erich Pfeiffer), Counting Pump™ (Ing. Erich Pfeiffer), Advanced Preservative Free System™ (Ing. Erich Pfeiffer), Unit Dose System™ (Ing. Erich Pfeiffer), Bidose System™ (Ing. Erich Pfeiffer), Bidose Powder System™ (Ing. Erich Pfeiffer), Sinus Science™ (Aerosol Science Laboratories, Inc., Camarillo, Calif.), ChiSys™ (Archimedes, Reading, UK), Fit-Lizer™ (Bioactis, Ltd, a SNBL subsidiary (Tokyo, J P), Swordfish V™ (Mystic Pharmaceuticals, Austin, Tex.), DirectHaler™ Nasal (DirectHaler, Copenhagen, Denmark) and SWIRLER™ Radioaerosol System (AMICI, Inc., Spring City, Pa.).

For administration via eye drops, a solution or suspension containing the caspase inhibitor or caspase inhibitor-cell penetrating peptide conjugate can be formulated for direct application to the retina by conventional means, for example with a dropper, pipette or spray. In certain embodiments, the caspase inhibitor or caspase inhibitor-cell penetrating peptide conjugate of the present disclosure is formulated in isotonic saline. In certain embodiments, the caspase inhibitor or caspase inhibitor-cell penetrating peptide conjugate of the present disclosure is formulated in isotonic saline at or about pH 7.4.

To facilitate delivery to a cell, tissue, or subject, the caspase inhibitors or caspase inhibitor-cell penetrating peptide conjugates of the present disclosure may, in various compositions, be formulated with a pharmaceutically-acceptable carrier, excipient, or diluent. The term “pharmaceutically-acceptable”, as used herein, means that the carrier, excipient, or diluent of choice does not adversely affect either the biological activity of the caspase inhibitors or caspase inhibitor-cell penetrating peptide conjugatesor the biological activity of the recipient of the composition. Suitable pharmaceutical carriers, excipients, and/or diluents for use in the present disclosure include, but are not limited to, lactose, sucrose, starch powder, talc powder, cellulose esters of alkonoic acids, magnesium stearate, magnesium oxide, crystalline cellulose, methyl cellulose, carboxymethyl cellulose, gelatin, glycerin, sodium alginate, gum arabic, acacia gum, sodium and calcium salts of phosphoric and sulfuric acids, polyvinylpyrrolidone and/or polyvinyl alcohol, saline, and water. Specific formulations of compounds for therapeutic treatment are discussed in Hoover, J. E., Remington's Pharmaceutical Sciences (Easton, Pa.: Mack Publishing Co., 1975) and Liberman and Lachman, eds., Pharmaceutical Dosage Forms (New York, N.Y.: Marcel Decker Publishers, 1980).

In accordance with the methods of the present disclosure, the quantity of the caspase inhibitors or caspase inhibitor-cell penetrating peptide conjugatesthat is administered to a cell, tissue, or subject should be an amount that is effective to inhibit the caspase within the tissue or subject. This amount is readily determined by the practitioner skilled in the art. The specific dosage employed in connection with any particular embodiment of the present disclosure will depend upon a number of factors, including the type inhibitor used, the caspase to be inhibited, and the cell type expressing the target. Quantities will be adjusted for the body weight of the subject, and the particular disease or condition being targeted.

### Methods of Treatment

In some embodiments, the high concentration caspase inhibitor-cell penetrating peptide conjugates may be used in methods of inhibiting apoptosis associated with ischemic injury in the CNS, ameliorating neurodegenerative diseases associated with apoptosis in the CNS, preventing or reducing inflammation, or inhibition of DME and/or RVO.

In certain embodiments, the instant disclosure is directed to methods of ameliorating the impact of CNS ischemic injury or decreasing the risk or manifestation of neurodegenerative disease in a patient. For example, in certain embodiments, the present disclosure is directed to methods of administering an effective amount of a caspase inhibitor-cell penetrating peptide conjugate to a patient to inhibit apoptosis associated with ischemic injury or apoptosis associated with neurodegenerative disorders. Accordingly, in some embodiments, the caspase inhibitor-cell penetrating peptide conjugate may be an apoptosis inhibitor cell penetrating peptide conjugate, as described in U.S. patent application Ser. No. 13/768,687, such as a cell penetrating peptide conjugate of XBIR3 or dominant negative caspase-6.

Accordingly, in certain embodiments, the methods of the instant disclosure are directed to the intranasal administration of a caspase inhibitor-cell penetrating peptide conjugate to inhibit apoptosis associated with ischemic injury in the central nervous system. In certain embodiments of the instant disclosure, the caspase inhibitor-cell penetrating peptide conjugate is administered during a treatment window that begins at the onset of ischemia and extends over the next 48 hours, where treatment is preferably administered within about 24 hours or within about 12 hours of the ischemic event.

In certain embodiments, the instant disclosure is directed to methods of preventing or reducing inflammation and optionally also preventing or reducing neurodegeneration resulting from inflammation in a patient by administering an effective amount of a caspase inhibitor-cell penetrating peptide conjugate. In some embodiments, the caspase inhibitor-cell penetrating peptide conjugate may be a caspase-9 signaling pathway inhibitor such as a caspase-9 inhibitor, a caspase-7 inhibitor, or an Apaf-1 inhibitor, as described in U.S. Provisional Patent Application No. 62/840,234. In some embodiments, to prevent or reduce the inflammation in a patient, the caspase inhibitor-cell penetrating peptide conjugate may be administered via injection, inhalation, or topical administration. Injection may be intravenous, intraocular, intraarterial, intracerebral, intracerebroventricular, or sub-tenon's injection. Topical administration may be via eye drops, or directly onto skin, or intranasally.

In certain embodiments, the instant disclosure is directed to methods of ameliorating the impact of and/or inhibiting the induction and/or exacerbation of DME and/or RVO in a patient by administering an effective amount of a caspase inhibitor-cell penetrating peptide conjugate. In some embodiments, the caspase inhibitor-cell penetrating peptide conjugate may be a caspase-9 signaling pathway inhibitor, such as those disclosed in U.S. patent application Ser. No. 16/243,884. In certain embodiments, the caspase inhibitor-cell penetrating peptide conjugate may be administered via eye drops in order to inhibit DME and/or RVO.

The caspase inhibitor-cell penetrating peptide conjugate may be administered as a single dose or multiple doses; where multiple doses are administered, they may be administered at intervals of 6 times per 24 hours or 4 times per 24 hours or 3 times per 24 hours or 2 times per 24 hours or 1 time per 24 hours or 1 time every other day or 1 time every 3 days or 1 time every 4 days or 1 time per week, or 2 times per week, or 3 times per week. The initial dose may be greater than subsequent doses or all doses may be the same.

In some embodiments of the instant disclosure, a caspase inhibitor-cell penetrating peptide conjugatemay be administered to a patient either as a single dose or in multiple doses. Where multiple doses are administered, they may be administered at intervals of 6 times per 24 hours or 4 times per 24 hours or 3 times per 24 hours or 2 times per 24 hours. The initial dose may be greater than subsequent doses or all doses may be the same.

In some embodiments, the concentration of the caspase inhibitor-cell penetrating peptide conjugate composition administered may be from, or from about, 0.5 mM to 5 mM, such as, for example 0.5 mM, 1 mM, 1.5 mM, 2 mM, 2.5 mM, 3 mM, 3.5 mM, 4 mM, 4.5 mM, or 5 mM. In some embodiments, a high concentration caspase inhibitor-cell penetrating peptide conjugate may be provided as described herein, and the concentration of the caspase inhibitor-cell penetrating peptide conjugate composition administered may be greater than 0.5 mM, such as, for example 0.5 mM, 1 mM, 1.5 mM, 2 mM, 2.5 mM, 3 mM, 3.5 mM, 4 mM, 4.5 mM, or 5 mM.

In some embodiments, the caspase inhibitor-cell penetrating peptide conjugate composition administered is composition may be delivered intranasally by administering, in certain embodiments, drops of 0.1 μl to 1000 μl; 1.0 μl to 500 μl; or 10 μl to 100 μl to alternating nares every 30 seconds to five minutes; every one minute to every four minutes; or every two minutes for 10 to 60 minutes; every 15 to 30 minutes; or every 20 minutes.

In some embodiments, a specific human equivalent dosage can be calculated from animal studies via body surface area comparisons, as outlined in Reagan-Shaw et al., FASEB J., 22; 659-661 (2007).

In some embodiments, eye size comparisons can be employed to calculate a specific human equivalent dosage, for example for administration of compositions for treating RVO or DME using eye drops.

In certain embodiments of the instant disclosure, the caspase inhibitor-cell penetrating peptide conjugate composition may be administered in conjunction with one or more additional therapeutics.

In some embodiments, the additional therapeutics include, but are not limited to, anticoagulant agents, such as tPA or heparin, free radical scavengers, anti-glutamate agents, etc. (see, for example, Zaleska et al., 2009, Neuropharmacol. 56(2):329-341). In certain embodiments the method involves the administration of one or more additional apoptotic target inhibitors either alone or in the context of a membrane-permeable complex. In some embodiments, the additional therapeutics include, but are not limited to, an anti-VEGF therapeutic and/or a steroidal therapeutic. In some embodiments, the method involves the administration of one or more additional caspase inhibitors.

As used herein, the term “patient” refers to any animal, including any mammal, including, but not limited to, humans, and non-human animals (including, but not limited to, non-human primates, dogs, cats, rodents, horses, cows, pigs, mice, rats, hamsters, rabbits, and the like. In particular, the patient is a human.

As used herein, an “effective amount” is an amount sufficient to cause a beneficial or desired clinical result in a patient. An effective amount can be administered to a patient in one or more doses. The effective amount is generally determined by the physician on a case-by-case basis and is within the skill of one in the art. Several factors may be taken into account when determining an appropriate dosage to achieve an effective amount. These factors include age, the condition being treated, the severity of the condition, prior responses, type of caspase inhibitor-cell penetrating peptide conjugate used, the type of target caspase to be inhibited, the cell type expressing the caspase target, and the form and effective concentration of the caspase inhibitor-cell penetrating peptide conjugate being administered.

In some embodiments, an effective amount is an amount that is sufficient to prevent or decrease apoptosis in a patient, including without limitation decreasing apoptosis associated with ischemia or neurodegenerative disease. In some embodiments, the effect amount ameliorates neurodegeneration resulting from apoptosis. In some embodiments, the neurodegenerative disease may be Alzheimer's Disease, Mild Cognitive Impairment, Parkinson's Disease, amyotrophic lateral sclerosis, Huntington's chorea, or Creutzfeld-Jacob disease.

In some embodiments, an “effective amount” is an amount sufficient to ameliorate the impact of and/or inhibit the induction and/or exacerbation of DME and/or RVO in a patient, or otherwise reduce the pathological consequences of the disease(s).

In some embodiments, an effective amount is an amount that is sufficient to prevent or decrease inflammation in a patient, including without limitation decreasing inflammation in a tissue of a patient, or systemically in a patient. In some embodiments, the effect amount may also ameliorate neurodegeneration resulting from inflammation. The effective amount is generally determined by the physician on a case-by-case basis and is within the skill of one in the art. Several factors may be taken into account when determining an appropriate dosage to achieve an effective amount. These factors include age, the condition being treated, the severity of the condition, prior responses, type of inhibitor used, the caspase-9 signaling pathway member to be inhibited, the cell type expressing the target, and the form and effective concentration of the composition (also referred to herein as a “treatment,” “inhibitor,” or “conjugate”) being administered.

Accordingly, in some embodiments, “treat,” “treating” and similar verbs refer to ameliorating, preventing or reducing inflammation and optionally ameliorating, preventing or reducing neurodegeneration resulting from inflammation in a patient.

Accordingly, in some embodiments, the compositions and methods described herein can be used for inhibiting a caspase-9 signaling pathway and thereby decreasing inflammation associated with various pathological conditions. For example, the compositions and methods described herein may be used for reducing retinal inflammation in diseases having an inflammatory component. In some embodiments, the term “neuroinflammation” refers to inflammation of a nervous system tissue. For example, such diseases include without limitation RVO, diabetic macular edema, retinal detachment, ocular trauma, retinitis pigmentosa, age-related macular degeneration (AMD), uveitis, retinal degenerative diseases, and glaucoma, among others described herein or identifiable by skilled persons upon reading the present disclosure. Exemplary retinal diseases include, without limitation, Leber's Hereditary Optic Neuropathy, Leigh Syndrome, Stargadt, retinitis pigmentosa, Best disease, and birdshot retinopathy, among others. The compositions and methods described herein may also be used for reducing ocular inflammation in non-hereditary inflammatory conditions including, without limitation, dry eye disease (conjunctivitis), episcleritis, and atopic dermatitis, among others. In some embodiments, the compositions and methods described herein can be used for treating neuroinflammatory injury in central nervous system (CNS) tissues, such as neuroinflammatory injury in multiple sclerosis, Behcets, Lupus, Systemic sarcoidosis, and central serous chorioretinopathy, among others. In some embodiments, the compositions and methods described herein can be used for treating ocular pathology in systemic neuroinflammatory diseases, such as Behcet's disease, multiple sclerosis, or systemic Lupus erythematosus, among others described herein or identifiable by skilled persons upon reading the present disclosure. In some embodiments, the compositions and methods described herein can be used for treating vasculitis. In some embodiments, the compositions and methods described herein can be used for reducing leukocyte infiltration into tissues, for example to treat sepsis, attenuate cytokine storm, or modulate innate immune response, among others described herein or identifiable by skilled persons upon reading the present disclosure. In some embodiments, the compositions and methods described herein can be used for treating inflammatory conditions including without limitation inflammatory bowel disease (IBD), rhegmatogenous retinal detachment, ischemic stroke, amyotrophic lateral sclerosis (ALS), or atherosclerosis, among others described herein or identifiable by skilled persons upon reading the present disclosure. In some embodiments, the compositions and methods described herein can be used to treat inflammatory consequences of XIAP deficiency disorder or lymphoproliferative syndrome.

### EXAMPLES

### Example 1

### Reduction of XBIR3 Using Dithiothreitol (DTT)

In the Examples disclosed in U.S. patent application Ser. No. 13/768,687, U.S. patent application Ser. No. 16/243,884, and U.S. Provisional Patent Application No. 62/840,234, using Pen1-XBIR3 in cell culture and mouse and rat studies, the Pen1-XBIR3 was produced using XBIR3 that had not been reduced using DTT. The Pen1-XBIR3 in the previous examples was produced in sufficient concentration for cell culture and mouse studies. Administration of pharmaceutical compositions in larger animals and humans typically requires production of pharmaceutical compositions in higher concentrations for improved efficacy.

XBIR3 contains cysteine residues, and the thiol side chains of the cysteine residues are susceptible to oxidation resulting in the disulfide derivative cystine, which can cause dimerization of XBIR3, and which may prevent efficient linkage of XBIR3 to a cell-penetrating peptide such as Penl, preventing production of disulfide linked Pen1-XBIR3 in sufficient concentration for improved efficacy in larger animals and humans.

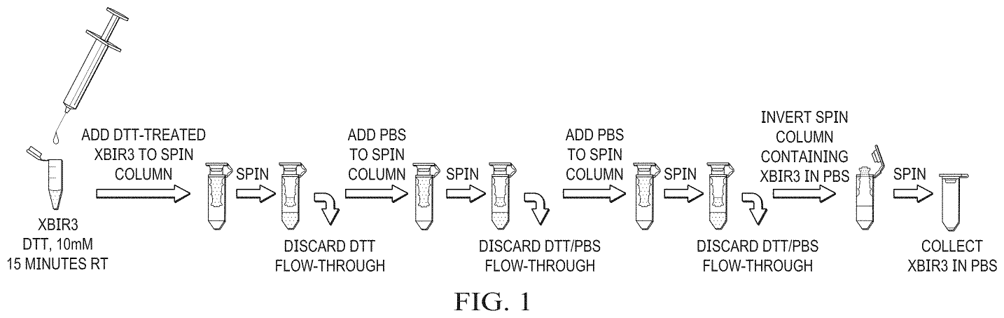

In the present Example, the BIR3 domain from XIAP (XBIR3) was purified as previously described (Sun, et al., J Biol Chem 275 (43), 33777-33781 (2000)). To decrease oxidation of the cysteine residues of XBIR3, keeping the cysteine residues in a reduced form to decrease dimerization of XBIR3, XBIR3 was incubated with dithiothreitol (DTT). As shown in schematic form in FIG. 1, XBIR3 was incubated in a tube with 10 mM DTT for 15 minutes at room temperature, in order to provide reduced XBIR3. Next, the solution containing the reduced XBIR3 and the DTT was transferred to a spin column placed in a collection tube (Amicon® Centrifugal Filter Unit, Merck, Germany, having a capacity of 0.5 mL and a 3K membrane). The Centrifugal Filter Unit was then centrifuged (centrifugation steps labeled as “Spin” in FIG. 1) at 10,000×g for 30 minutes at room temperature to remove excess DTT. The 3K membranes of the Centrifugal Filter Unit allow DTT and Phosphate Buffered Saline (PBS) to flow through and prevent flow-through of the reduced XBIR3. Approximately 50 μL XBIR3 solution remained in the spin column after centrifugation. The DTT flow-through in the collection tube was then discarded.

Next, for buffer exchange of the reduced XBIR3 into PBS, removing DTT, a first volume of 450 μL PBS was added to the approximately 50 μL reduced XBIR3 in the spin column. The Centrifugal Filter Unit was centrifuged at 10,000×g for 30 minutes at room temperature. Approximately 50 μL XBIR3 solution remained in the spin column after centrifugation. The PBS and DTT flow-through in the collection tube was then discarded.

Next, a second volume of 450 μL PBS was added to the approximately 50 μL reduced XBIR3 in the spin column. The Centrifugal Filter Unit was centrifuged at 10,000×g for 30 minutes at room temperature. Approximately 50 μL XBIR3 solution remained in the spin column after centrifugation. The PBS and DTT flow-through in the collection tube was then discarded.

Next, the spin column containing the remaining approximately 50 μL of solution containing the reduced XBIR3 in PBS was inverted and placed in the collection tube. The Centrifugal Filter Unit was then centrifuged to recover the reduced XBIR3 in the collection tube. For collection of the reduced XBIR3 in PBS, the inverted column was centrifuged in the collection tube at 10,000×g for 2 minutes.

### Example 2

### PAGE Analysis of DTT-Reduced XBIR3

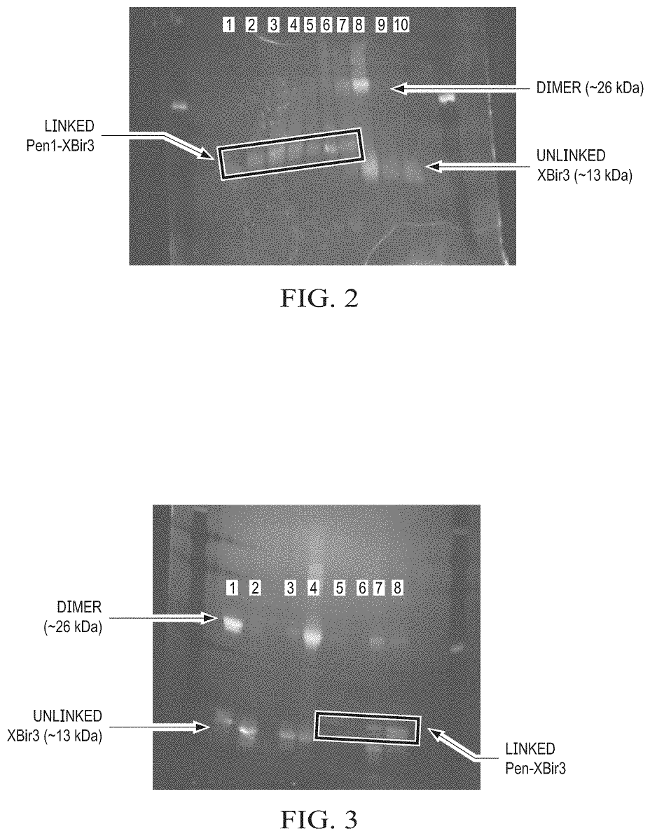

FIG. 2 shows an image visualizing a stained 20% PAGE (polyacrylamide gel electrophoresis) gel after PAGE separation of samples of linked Pent-XBIR3 that were prepared using a range of concentrations of DTT-treated (reduced) XBIR3. Lanes 1-6 show XBIR3 that was reduced with DTT and was successfully linked to Penl at increasing concentrations 150 uM, 500 uM, 3.1 mM, and 1.4 mM, specifically 150 μM XBIR3 (lanes 1 and 2), 500 μM XBIR3 (lanes 3 and 4), 3.1 mM XBIR3 (lane 5), and 1.4 mM XBIR3 (lane 6). The Pen1-XBIR3 samples shown in lanes 1-6 were prepared using XBIR3 that had been reduced with DTT as described in Example 1. There are no XBIR3 dimers present for the Pen1-XBIR3 samples in lanes 1-6.

Lane 7 shows a linked Pen1-XBIR3 sample that was prepared using 160 μM XBIR3 that had not been reduced with DTT. In contrast with the DTT-treated samples in lanes 1-6, the sample in lane 7 shows an XBIR3 dimer at ˜26 kDa.

Equal quantities of Pen1-XBIR3 were loaded for each of lanes 1-7, thus allowing a comparison of linked vs. homo-dimerized XBIR3 at the indicated concentrations and conditions.

Lanes 8-10 show XBIR3, not linked to Penl, in which the XBIR3 sample shown in lane 8 was not reduced with DTT. In contrast, the samples shown in lane 9 shows 3.1 mM XBIR3 that has been reduced with DTT as described in Example 1 and lane 10 shows 1.4 mM XBIR3 that has been reduced with DTT as described in Example 1.

The sample shown in lane 8, in which XBIR3 had not been reduced with DTT, shows a dimer of XBIR3 at approximately 26 kDa, in addition to a monomer of XBIR3 at approximately 13 kDa. In contrast, the samples shown in lanes 9 and 10, in which XBIR3 had been reduced with DTT as described in Example 1, shows a monomer of XBIR3 at approximately 13 kDa, however a dimer of XBIR3 at approximately 26 kDa, is negligible or absent. This Example shows that reducing XBIR3 with DTT prevents or reduces XBIR3 dimer formation and allows Pen-1XBIR3 linkage at higher concentrations.

### Example 3

### PAGE Analysis of Linked Pen1-XBIR3 Produced Using DTT-Reduced XBIR3

Penetratin1 (Pen1, Q-Biogene, Carlsbad, CA) was mixed at an equimolar ratio with purified XBIR3, with or without DTT treatment of the XBIR3 as in Example 1, and incubated for 30 minutes at room temperature to generate disulfide-linked Pen1-BIR3.

FIG. 3 shows an image visualizing a stained 20% PAGE (polyacrylamide gel electrophoresis) gel after PAGE separation of samples of XBIR3 with or without DTT treatment, and linked Pen1-XBIR3 produced with or without DTT treatment of the XBIR3.

In particular, lane 1 shows XBIR3 without DTT treatment, showing a monomer of XBIR3 at approximately 13 kDa as well as a dimer of XBIR3 at approximately 26 kDa. Lane 2 shows reduced XBIR3 after DTT treatment as described in Example 1, showing a monomer of XBIR3 at approximately 13 kDa, whereas a dimer of XBIR3 at 26 kDA is negligible or absent following DTT treatment of XBIR3. Lane 3 shows DTT-reduced XBIR3 following buffer exchange into PBS as described in Example 1. Similar to lane 1, lane 4 shows XBIR3 without treatment with DTT, showing a monomer of XBIR3 at approximately 13 kDa as well as a dimer of XBIR3 at approximately 26 kDa. Lane 5 shows Pen1-XBIR3 produced using 500 μM XBIR3 without DTT treatment, showing a faint band corresponding to linked Pen1-XBIR3. Lane 6 shows 500 μM unlinked XBIR3 without DTT treatment. In contrast, lanes 7 and 8 show a stronger band corresponding to linked Pen1-XBIR3 produced using 500 XBIR3 μM that had been reduced using DTT. These results show that a higher concentration of Pen1-XBIR3 is produced using XBIR3 reduced using DTT.

Linking the reduced XBIR3 to the Penl to provide the linked Pen1-XBIR3 was performed using equimolar (1:1) reduced XBIR3 : Penl. Linkage was typically performed from 0 to 30 minutes after the last buffer exchange step. Concentration of reduced XBIR3 was determined by UV spectrophotometry (Nanodrop®, NanoDrop Technologies, LLC). Linkage of reduced XBIR3 to Penl was performed for 30 minutes at room temperature.

By reducing XBIR3 using DTT as described in Example 1, linkage of up to 3.1 mM Pen1-XBIR3 was achieved, providing Pen1-XBIR3 in sufficient concentration for improved efficacy in larger animals and humans.

### Example 4

### Circular Dichroism Structural Analysis of XBIR3 and Pen1-XBIR3

The molecular structures of XBIR3 and Pen1-XBIR3 were analyzed using circular dichroism.

Circular dichroism data were collected for XBIR3 and Pen1-XBIR3 at 3 temperatures (4° C., 23° C. and 34° C.) and at two concentrations (0.2 mg/ml and 0.3 mg/ml). Circular dichroism data were obtained using a ChiraScan V100 CD spectrometer. Each sample had data obtained from three independent scans at the indicated temperature setting which were averaged together. Each sample also had its own blank control in order to subtract the background signal of the buffer.

The circular dichroism data of XBIR3 and Pen1-XBIR3 were analyzed using the BeStSel (Beta. Structure Selection) web server at http://bestsel.elte.hu according to the methods described by Micsonai et al. (2018) Nucleic Acids Research 46:W315-22 and Micsonai et al. (2015) Proc. Acad. Sci. U.S.A. 11:E3095-103, the disclosures of which are incorporated by reference in their entireties.

The wavelength range was 200-250 nm and a scale factor of 1 was used.

For the secondary structure determination, The BeStSel web server uses pre-calculated, fixed basis spectrum sets by fitting the CD spectrum of any unknown protein with the linear combination of the basis spectra. Graphs of the fitting results for each sample were reported.

The root-mean-square deviation (RMSD) of secondary structure and also the normalized RMSD (NRMSD) were also estimated according to the methods described in Micsonai et al. (2018) Nucleic Acids Research 46:W315-22 and Micsonai et al. (2015) Proc. Acad. Sci. U.S.A. 11:E3095-103.

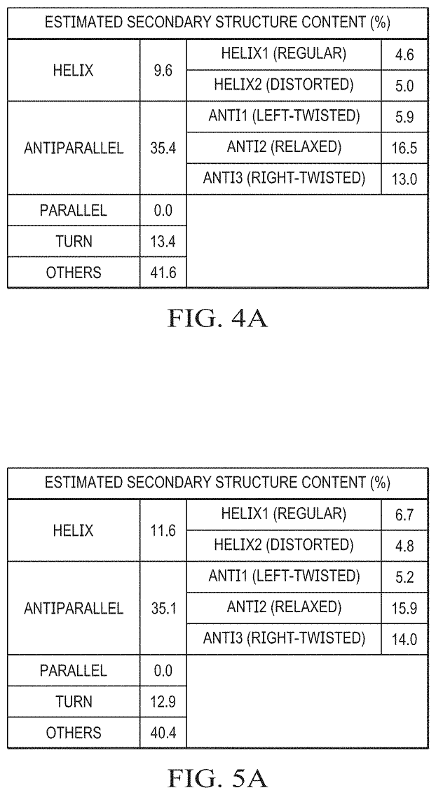

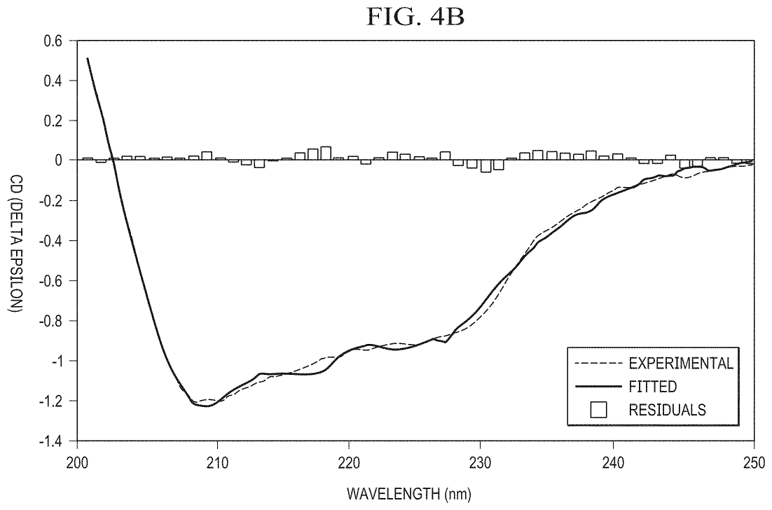

The secondary structure content of XBIR3 at a concentration of 0.3 mg/ml and a temperature of 23° C. is reported in FIG. 4A, and a graph reporting the fitting results is shown in FIG. 4B. The RMSD of secondary structure was 0.0259 and the NRMSD was 0.01517.

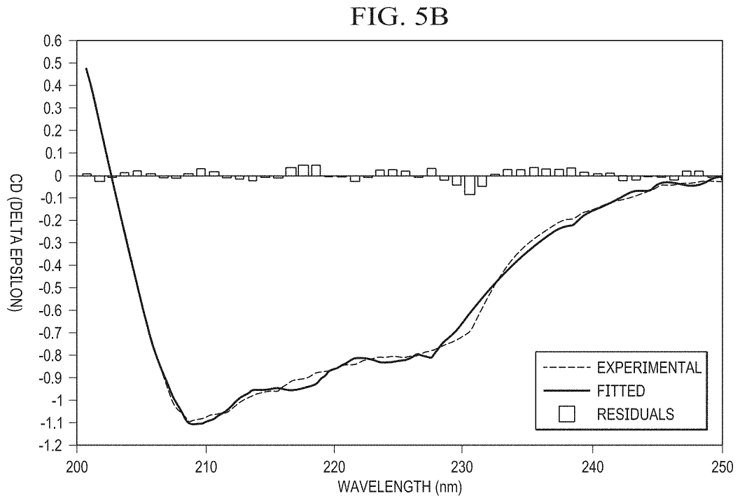

The secondary structure content of Pen1-XBIR3 at a concentration of 0.3 mg/ml and a temperature of 23° C. is reported in FIG. 5A, and a graph reporting the fitting results is shown in FIG. 5B, The RMSD of secondary structure was 0.0255 and the NRMSD was 0.01557.

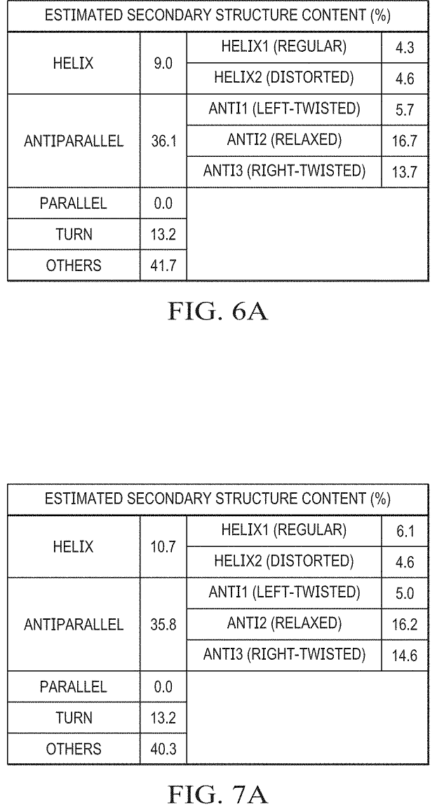

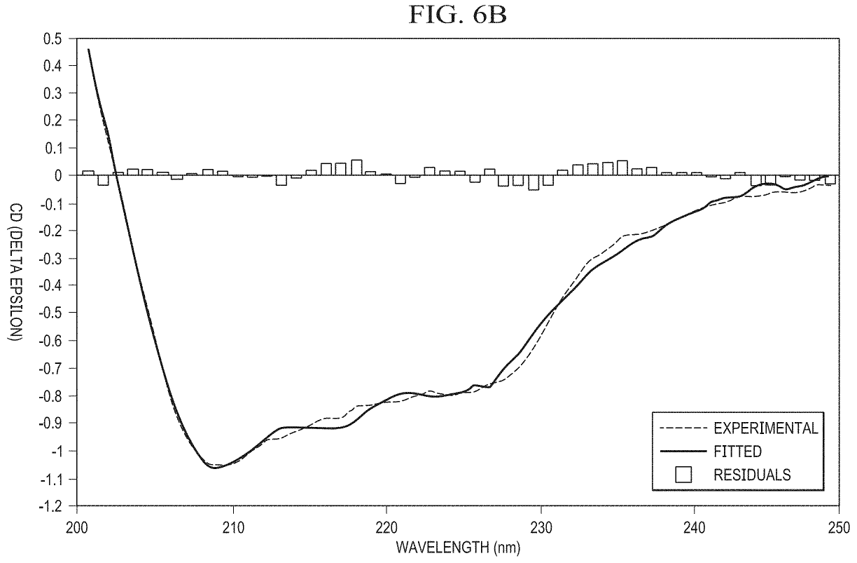

The secondary structure content of XBIR3 at a concentration of 0.3 mg/ml and a temperature of 4° C. is reported in FIG. 6A, and a graph reporting the fitting results is shown in FIG. 6B. The RMSD of secondary structure was 0.0265 and the NRMSD was 0.01684.

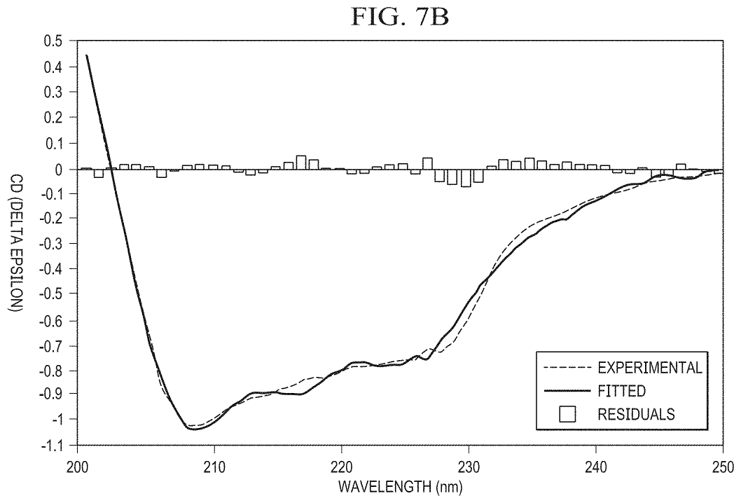

The secondary structure content of Pen1-XBIR3 at a concentration of 0.3 mg/ml and a temperature of 4° C. is reported in FIG. 7A, and a graph reporting the fitting results is shown in FIG. 7B. The RMSD of secondary structure was 0.0265 and the NRMSD was 0.01725.

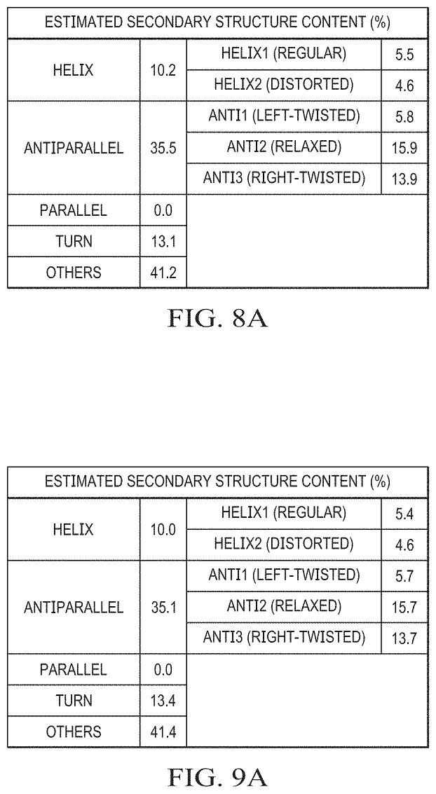

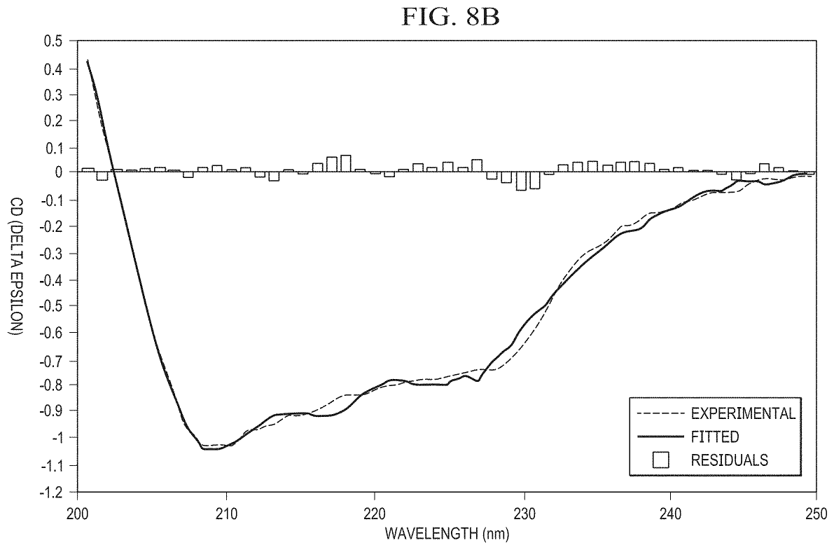

The secondary structure content of XBIR3 at a concentration of 0.3 mg/ml and a temperature of 34° C. is reported in FIG. 8A, and a graph reporting the fitting results is shown in FIG. 8B. The RMSD of secondary structure was 0.0265 and the NRMSD was 0.01763.

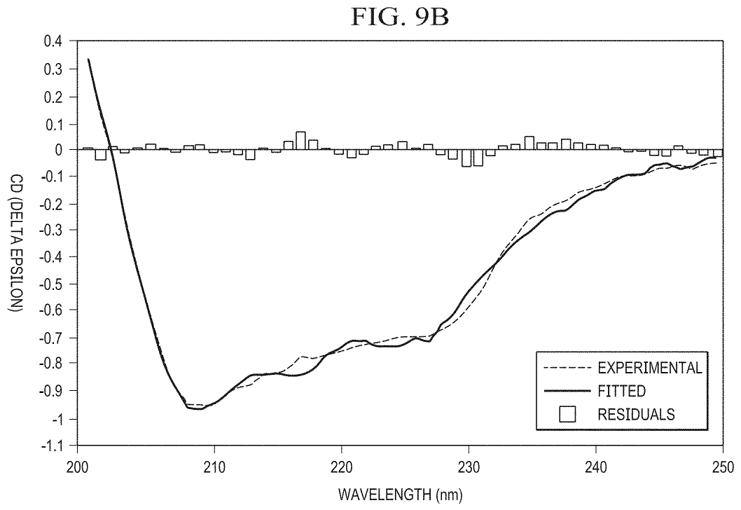

The secondary structure content of Pen1-XBIR3 at a concentration of 0.3 mg/ml and a temperature of 34° C. is reported in FIG. 9A, and a graph reporting the fitting results is shown in FIG. 9B. The RMSD of secondary structure was 0.026 and the NRMSD was 0.0191.

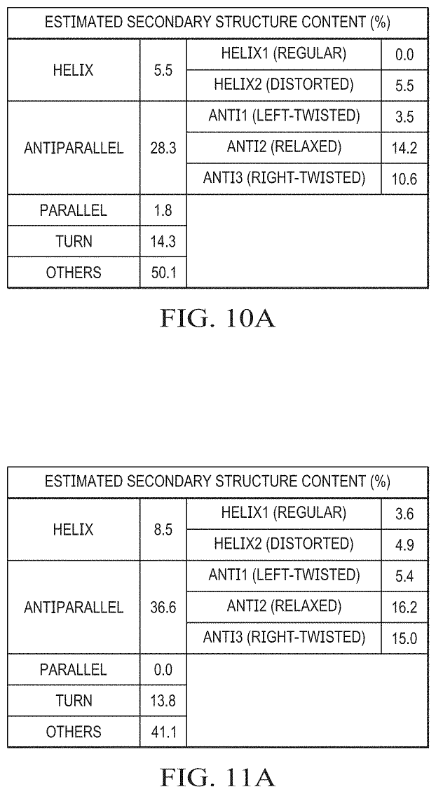

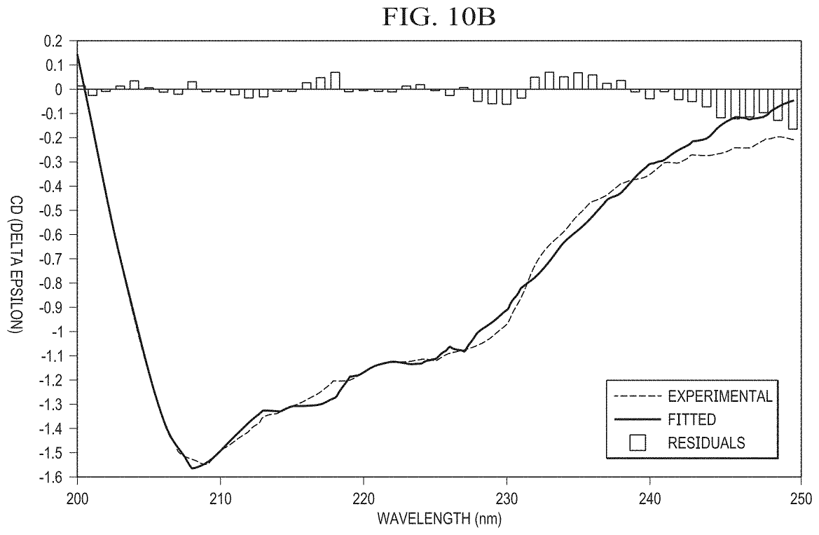

The secondary structure content of XBIR3 at a concentration of 0.2 mg/ml and a temperature of 23° C. is reported in FIG. 10A, and a graph reporting the fitting results is shown in FIG. 10B. The RMSD of secondary structure was 0.0556 and the NRMSD was 0.03243.

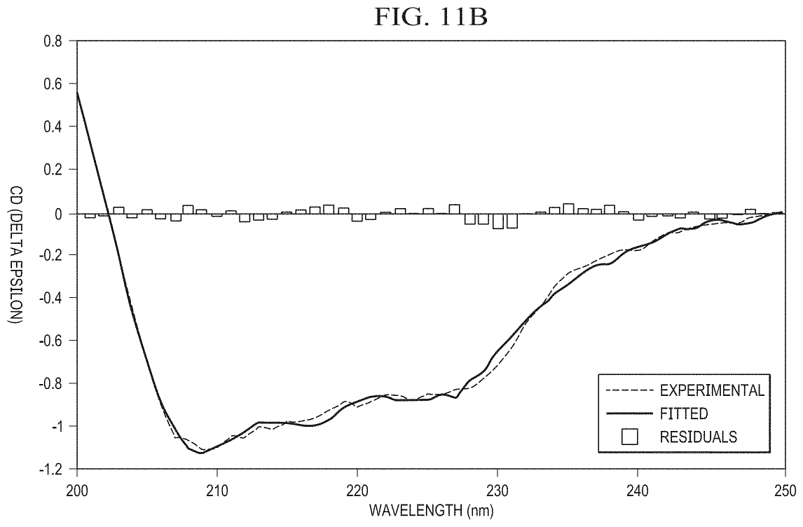

The secondary structure content of Pen1-XBIR3 at a concentration of 0.2 mg/ml and a temperature of 23° C. is reported in FIG. 11A, and a graph reporting the fitting results is shown in FIG. 11B The RMSD of secondary structure was 0.0261 and the NRMSD was 0.01551.

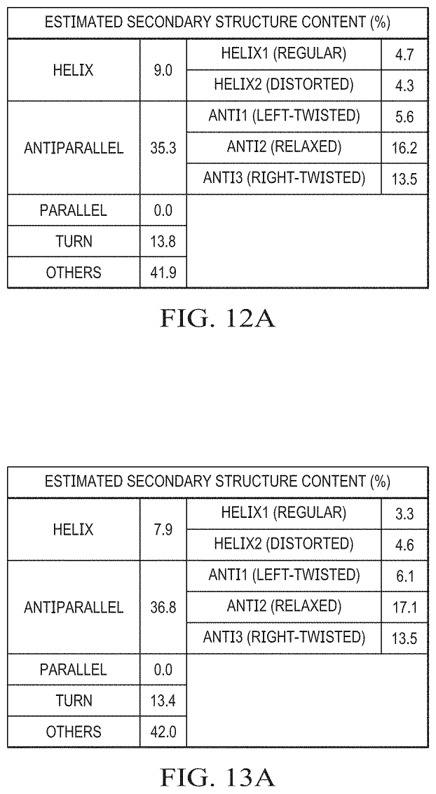

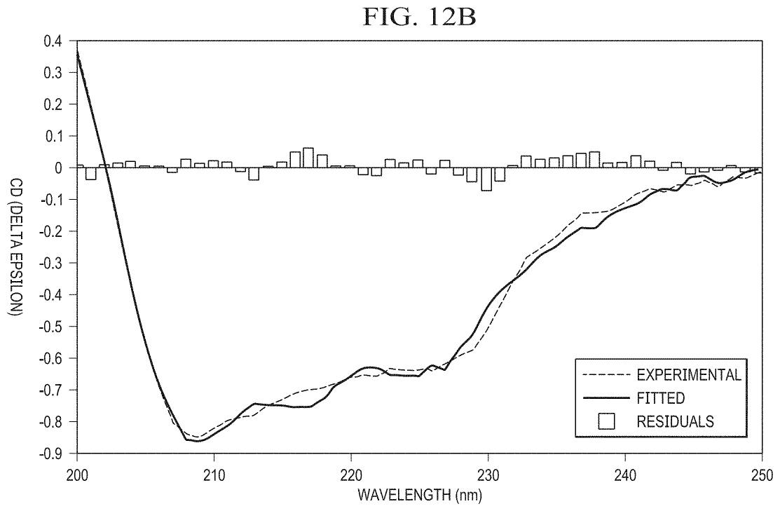

The secondary structure content of XBIR3 at a concentration of 0.2 mg/ml and a temperature of 4° C. is reported in FIG. 12A, and a graph reporting the fitting results is shown in FIG. 12B. The RMSD of secondary structure was 0.0263 and the NRMSD was 0.02152.

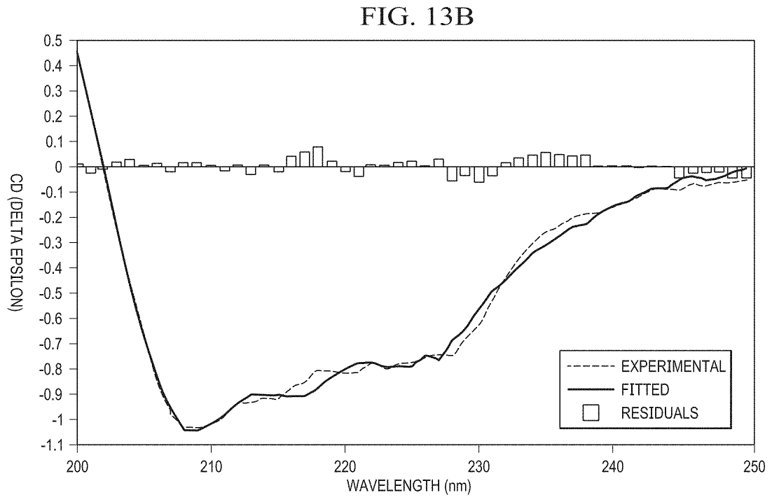

The secondary structure content of Pen1-XBIR3 at a concentration of 0.2 mg/ml and a temperature of 4° C. is reported in FIG. 13A, and a graph reporting the fitting results is shown in FIG. 13B. The RMSD of secondary structure was 0.0294 and the NRMSD was 0.01974.

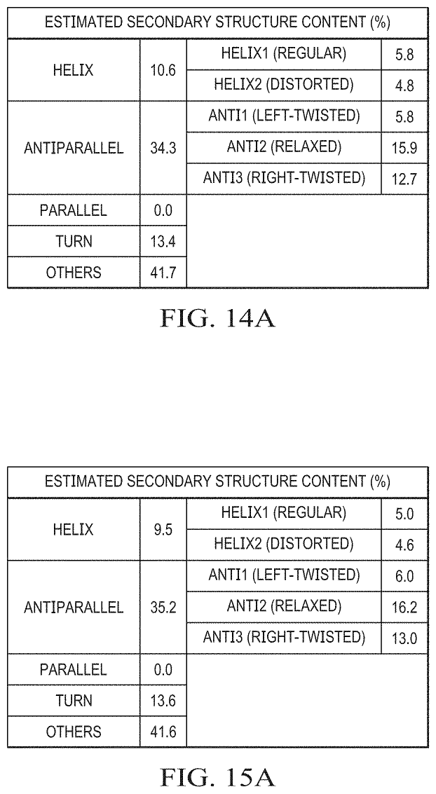

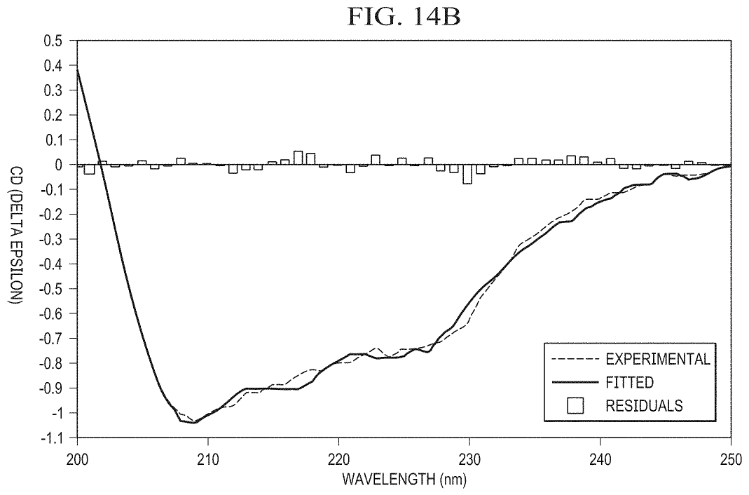

The secondary structure content of XBIR3 at a concentration of 0.2 mg/ml and a temperature of 34° C. is reported in FIG. 14A, and a graph reporting the fitting results is shown in FIG. 14B. The RMSD of secondary structure was 0.0239 and the NRMSD was 0.01678.

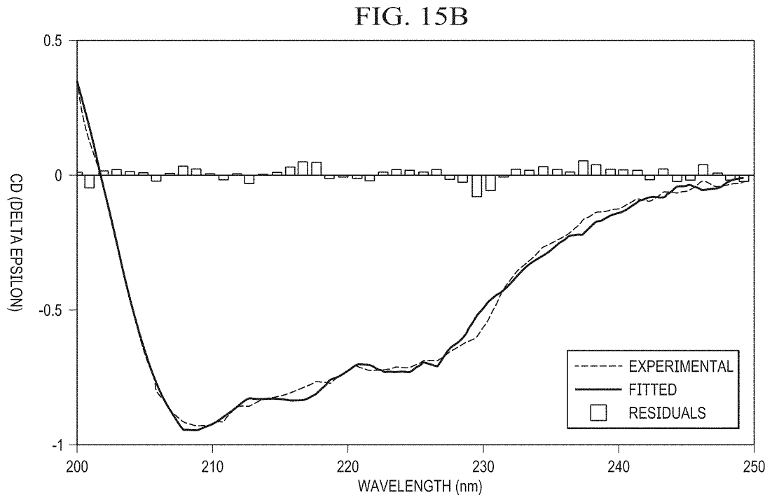

The secondary structure content of Pen1-XBIR3 at a concentration of 0.2 mg/ml and a temperature of 34° C. is reported in FIG. 15A, and a graph reporting the fitting results is shown in FIG. 15B. The RMSD of secondary structure was 0.0255 and the NRMSD was 0.01967.

The circular dichroism results show that the secondary structure of both XBIR3 and Pen1-XBIR3 is composed of approximately 10% helix and approximately 35% anti-parallel β-sheet. Thus, linkage of XBIR3 to Penl did not markedly change the XBIR3 secondary structure. Furthermore, the secondary structure was not markedly changed by changes in temperature or concentration.

Various publications are cited herein, the contents of which are hereby incorporated in their entireties.

Amino Acid Sequence: c-IAP1 (Accession No. Q13490.2):
(SEQ ID NO: 45)
MHKTASQRLFPGPSYQNIKSIMEDSTILSDWTNSNKQKMKYDFSCELYRMSTYSTFPAG
VPVSERSLARAGFYYTGVNDKVKCFCCGLMLDNWKLGDSPIQKHKQLYPSCSFIQNLVS
ASLGSTSKNTSPMRNSFAHSLSPTLEHSSLFSGSYSSLSPNPLNSRAVEDISSSRTNPYSYA
MSTEEARFLTYHMWPLTFLSPSELARAGFYYIGPGDRVACFACGGKLSNWEPKDDAMS
EHRRHFPNCPFLENSLETLRFSISNLSMQTHAARMRTFMYWPSSVPVQPEQLASAGFYY
VGRNDDVKCFCCDGGLRCWESGDDPWVEHAKWFPRCEFLIRMKGQEFVDEIQGRYPH
LLEQLLSTSDTTGEENADPPIIHFGPGESSSEDAVMMNTPVVKSALEMGFNRDLVKQTV
QSKILTTGENYKTVNDIVSALLNAEDEKREEEKEKQAEEMASDDLSLIRKNRMALFQQL
TCVLPILDNLLKANVINKQEHDIIKQKTQIPLQARELIDTILVKGNAAANIFKNCLKEIDST
LYKNLFVDKNMKYIPTEDVSGLSLEEQLRRLQEERTCKVCMDKEVSVVFIPCGHLVVCQ
ECAPSLRKCPICRGIIKGTVRTFLS
Amino Acid Sequence: c-IAP2 (Accession No. Q13489.2):
(SEQ ID NO: 46)
MNIVENSIFLSNLMKSANTFELKYDLSCELYRMSTYSTFPAGVPVSERSLARAGFYYTGV
NDKVKCFCCGLMLDNWKRGDSPTEKHKKLYPSCRFVQSLNSVNNLEATSQPTFPSSVT
NSTHSLLPGTENSGYFRGSYSNSPSNPVNSRANQDFSALMRSSYHCAMNNENARLLTFQ
TWPLTFLSPTDLAKAGFYYIGPGDRVACFACGGKLSNWEPKDNAMSEHLRHFPKCPFIE
NQLQDTSRYTVSNLSMQTHAARFKTFFNWPSSVLVNPEQLASAGFYYVGNSDDVKCFC
CDGGLRCWESGDDPWVQHAKWFPRCEYLIRIKGQEFIRQVQASYPHLLEQLLSTSDSPG
DENAESSIIHFEPGEDHSEDAIMMNTPVINAAVEMGFSRSLVKQTVQRKILATGENYRLV
NDLVLDLLNAEDEIREEERERATEEKESNDLLLIRKNRMALFQHLTCVIPILDSLLTAGIIN
EQEHDVIKQKTQTSLQARELIDTILVKGNIAATVFRNSLQEAEAVLYEHLFVQQDIKYIPT
EDVSDLPVEEQLRRLQEERTCKVCMDKEVSIVFIPCGHLVVCKDCAPSLRKCPICRSTIKG
TVRTFLS
Amino Acid Sequence: XIAP (Accession No. P98170.2):
(SEQ ID NO: 47)
MTFNSFEGSKTCVPADINKEEEFVEEFNRLKTFANFPSGSPVSASTLARAGFLYTGEGDT
VRCFSCHAAVDRWQYGDSAVGRHRKVSPNCRFINGFYLENSATQSTNSGIQNGQYKVE
NYLGSRDHFALDRPSETHADYLLRTGQVVDISDTIYPRNPAMYSEEARLKSFQNWPDYA
HLTPRELASAGLYYTGIGDQVQCFCCGGKLKNWEPCDRAWSEHRRHFPNCFFVLGRNL
NIRSESDAVSSDRNFPNSTNLPRNPSMADYEARIFTFGTWIYSVNKEQLARAGFYALGEG
DKVKCFHCGGGLTDWKPSEDPWEQHAKWYPGCKYLLEQKGQEYINNIHLTHSLEECLV
RTTEKTPSLTRRIDDTIFQNPMVQEAIRMGFSFKDIKKIMEEKIQISGSNYKSLEVLVADL
VNAQKDSMQDESSQTSLQKEISTEEQLRRLQEEKLCKICMDRNIAIVFVPCGHLVTCKQC
AEAVDKCPMCYTVITFKQKIFMS
Amino Acid Sequence: NAIP (Accession No. Q13075.3):
(SEQ ID NO: 48)
MATQQKASDERISQFDHNLLPELSALLGLDAVQLAKELEEEEQKERAKMQKGYNSQMR
SEAKRLKTFVTYEPYSSWIPQEMAAAGFYFTGVKSGIQCFCCSLILFGAGLTRLPIEDHKR
FHPDCGFLLNKDVGNIAKYDIRVKNLKSRLRGGKMRYQEEEARLASFRNWPFYVQGISP
CVLSEAGFVFTGKQDTVQCFSCGGCLGNWEEGDDPWKEHAKWFPKCEFLRSKKSSEEI
TQYIQSYKGFVDITGEHFVNSWVQRELPMASAYCNDSIFAYEELRLDSFKDWPRESAVG
VAALAKAGLFYTGIKDIVQCFSCGGCLEKWQEGDDPLDDHTRCFPNCPFLQNMKSSAE
VTPDLQSRGELCELLETTSESNLEDSIAVGPIVPEMAQGEAQWFQEAKNLNEQLRAAYTS
ASFRHMSLLDISSDLATDHLLGCDLSIASKHISKPVQEPLVLPEVFGNLNSVMCVEGEAG
SGKTVLLKKIAFLWASGCCPLLNRFQLVFYLSLSSTRPDEGLASIICDQLLEKEGSVTEMC
VRNIIQQLKNQVLFLLDDYKEICSIPQVIGKLIQKNHLSRTCLLIAVRTNRARDIRRYLETI
LEIKAFPFYNTVCILRKLFSHNMTRLRKFMVYFGKNQSLQKIQKTPLFVAAICAHWFQYP
FDPSFDDVAVFKSYMERLSLRNKATAEILKATVSSCGELALKGFFSCCFEFNDDDLAEAG
VDEDEDLTMCLMSKFTAQRLRPFYRFLSPAFQEFLAGMRLIELLDSDRQEHQDLGLYHL
KQINSPMMTVSAYNNFLNYVSSLPSTKAGPKIVSHLLHLVDNKESLENISENDDYLKHQP
EISLQMQLLRGLWQICPQAYFSMVSEHLLVLALKTAYQSNTVAACSPFVLQFLQGRTLT
LGALNLQYFFDHPESLSLLRSIHFPIRGNKTSPRAHFSVLETCFDKSQVPTIDQDYASAFEP
MNEWERNLAEKEDNVKSYMDMQRRASPDLSTGYWKLSPKQYKIPCLEVDVNDIDVVG
QDMLEILMTVFSASQRIELHLNHSRGFIESIRPALELSKASVTKCSISKLELSAAEQELLLT
LPSLESLEVSGTIQSQDQIFPNLDKFLCLKELSVDLEGNINVFSVIPEEFPNFHHMEKLLIQI
SAEYDPSKLVKLIQNSPNLHVFHLKCNFFSDFGSLMTMLVSCKKLTEIKFSDSFFQAVPF
VASLPNFISLKILNLEGQQFPDEETSEKFAYILGSLSNLEELILPTGDGIYRVAKLIIQQCQQ
LHCLRVLSFFKTLNDDSVVEIAKVAISGGFQKLENLKLSINHKITEEGYRNFFQALDNMP
NLQELDISRHFTECIKAQATTVKSLSQCVLRLPRLIRLNMLSWLLDADDIALLNVMKERH
PQSKYLTILQKWILPFSPIIQK
Amino Acid Sequence: survivin (Accession No. O15392.2):
(SEQ ID NO: 49)
MGAPTLPPAWQPFLKDHRISTFKNWPFLEGCACTPERMAEAGFIHCPTENEPDLAQCFFC
FKELEGWEPDDDPIEEHKKHSSGCAFLSVKKQFEELTLGEFLKLDRERAKNKIAKETNN
KKKEFEETAEKVRRAIEQLAAMD
Amino Acid Sequence: BRUCE (Accession No. Q9118B7):
(SEQ ID NO: 50)
MSQILSALGLCNSSAMAMIIGASGLHLTKHENFHGGLDAISVGDGLFTILTTLSKKASTV
HMMLQPILTYMACGYMGRQGSLATCQLSEPLLWFILRVLDTSDALKAFHDMGGVQLIC
NNMVTSTRAIVNTAKSMVSTIMKFLDSGPNKAVDSTLKTRILASEPDNAEGIHNFAPLGT
ITSSSPTAQPAEVLLQATPPHRRARSAAWSYIFLPEEAWCNLTIHLPAAVLLKEIHIQPHL
ASLATCPSSVSVEVSADGVNMLPLSTPVVTSGLTYIKIQLVKAEVASAVCLRLHRPRDAS
TLGLSQIKLLGLTAFGTTSSATVNNPFLPSEDQVSKTSIGWLRLLHHCLTHISDLEGMMA
SAAAPTANLLQTCAALLMSPYCGMHSPNIEVVLVKIGLQSTRIGLKLIDILLRNCAASGS
DPTDLNSPLLFGRLNGLSSDSTIDILYQLGTSQDPGTKDRIQALLKWVSDSARVAAMKRS
GRMNYMCPNSSTVEYGLLMPSPSHLHCVAAILWHSYELLVEYDLPALLDQELFELLFN
WSMSLPCNMVLKKAVDSLLCSMCHVHPNYFSLLMGWMGITPPPVQCHHRLSMTDDSK
KQDLSSSLTDDSKNAQAPLALTESHLATLASSSQSPEAIKQLLDSGLPSLLVRSLASFCFS
HISSSESIAQSIDISQDKLRRHHVPQQCNKMPITADLVAPILRFLTEVGNSHIMKDWLGGS
EVNPLWTALLFLLCHSGSTSGSHNLGAQQTSARSASLSSAATTGLTTQQRTAIENATVAF
FLQCISCHPNNQKLMAQVLCELFQTSPQRGNLPTSGNISGFIRRLFLQLMLEDEKVTMFL
QSPCPLYKGRINATSHVIQHPMYGAGHKFRTLHLPVSTTLSDVLDRVSDTPSITAKLISEQ
KDDKEKKNHEEKEKVKAENGFQDNYSVVVASGLKSQSKRAVSATPPRPPSRRGRTIPDK
IGSTSGAEAANKIITVPVFHLFHKLLAGQPLPAEMTLAQLLTLLYDRKLPQGYRSIDLTVK
LGSRVITDPSLSKTDSYKRLHPEKDHGDLLASCPEDEALTPGDECMDGILDESLLETCPIQ
SPLQVFAGMGGLALIAERLSMLYPEVIQQVSAPVVTSTTLEKPKDSDQFEWVTIEQSGEL
VYEAPETVAAEPPPIKSAVQTMSPIPAHSLAAFGLFLRLPGYAEVLLKERKHAQCLLRLV
LGVTDDGEGSHILQSPSANVLPTLPFHVLRSLFSTTPLTTDDGVLLRRMALEIGALHLILV
CLSALSHHSPRVPNSSVNQTEPQVSSSHNPTSTEEQQLYWAKGTGFGTGSTASGWDVEQ
ALTKQRLEEEHVTCLLQVLASYINPVSSAVNGEAQSSHETRGQNSNALPSVLLELLSQSC
LIPAMSSYLRNDSVLDMARHVPLYRALLELLRAIASCAAMVPLLLPLSTENGEEEEEQSE
CQTSVGTLLAKMKTCVDTYTNRLRSKRENVKTGVKPDASDQEPEGLTLLVPDIQKTAEI
VYAATTSLRQANQEKKLGEYSKKAAMKPKPLSVLKSLEEKYVAVMKKLQFDTFEMVS
EDEDGKLGFKVNYHYMSQVKNANDANSAARARRLAQEAVTLSTSLPLSSSSSVFVRCD
EERLDIMKVLITGPADTPYANGCFEFDVYFPQDYPSSPPLVNLETTGGHSVRFNPNLYND
GKVCLSILNTWHGRPEEKWNPQTSSFLQVLVSVQSLILVAEPYFNEPGYERSRGTPSGTQ
SSREYDGNIRQATVKWAMLEQIRNPSPCFKEVIHKHFYLKRVEIMAQCEEWIADIQQYSS
DKRVGRTMSHHAAALKRHTAQLREELLKLPCPEDLDPDTDDAPEVCRATTGAEETLMH
DQVKPSSSKELPSDFQL

1. A method of providing a disulfide-linked caspase inhibitor-cell penetrating peptide conjugate, comprising:
incubating a caspase inhibitor having one or more thiol groups with a reducing agent selected from dithiothreitol (DTT), 2-mercaptoethanol (2-ME) and tris(2-carboxyethyl)phosphine (TCEP) to provide a reduced caspase inhibitor;
removing the reducing agent from the reduced caspase inhibitor; and
conjugating the reduced caspase inhibitor with a cell-penetrating peptide by a disulfide linkage.

2. The method of claim 1, wherein the caspase inhibitor is selected from the group consisting of a caspase -1, -2, -3, -4, -5, -6, -7, -8, -9, -10, -11, -12, and -14 inhibitor.

3. The method of claim 1, wherein the caspase inhibitor is selected from a XBIR3, a XBIR2, a linker-BIR2 and a dominant-negative caspase 6.

4. The method of claim 1, wherein the removing the reducing agent is by filtration.

5. The method of claim 1, further comprising a buffer exchange wherein the reduced caspase inhibitor is comprised in a pharmaceutically acceptable excipient.

6. The method of claim 1, wherein the cell-penetrating peptide is selected from Penetratin1, transportan, pIsl, TAT(48-60), pVEC, MTS, MAP, polyarginines, DPV1047, M918, M1073, BPrPr (1-28), MPG, Pep-1, MAP12, MAP17, GALA, p28, PreS2, VT5, Bac 7 [Bac (1-24)], PPR, PRR, SAP, SAP(E), CyLoP-1, gH 625, CPP-C, C105Y, Pep-7, and SG3.

7. The method of claim 1, wherein the caspase inhibitor-cell penetrating peptide conjugate is a disulfide-linked Penetratin1-XBIR3.

8. The method of claim 1, wherein the reduced caspase inhibitor has no more than 40% caspase inhibitor dimers.

9. The method of claim 1, wherein the reduced caspase inhibitor has at least 3-fold less dimer formation than the caspase inhibitor that has not been treated with the reducing agent.

10. The method of claim 1, wherein the caspase inhibitor-cell penetrating peptide conjugate has a concentration greater than 1 mM.

11. A composition comprising a disulfide linked caspase inhibitor-cell penetrating peptide conjugate and a pharmaceutically acceptable carrier, wherein the caspase inhibitor-cell penetrating peptide conjugate has a concentration greater than 1 mM.

12. The composition of claim 11, wherein the caspase inhibitor-cell penetrating peptide conjugate comprises a caspase inhibitor selected from the group consisting of a caspase -1, -2, -3, -4, -5, -6, -7, -8, -9, -10, -11, -12, and -14 inhibitor.

13. The composition of claim 11, wherein the caspase inhibitor is selected from a XBIR3, a XBIR2, a linker-BIR2 and a dominant-negative caspase 6.

14. The composition of claim 11, wherein the cell-penetrating peptide is selected from Penetratin1, transportan, pIsl, TAT(48-60), pVEC, MTS, MAP, polyarginines, DPV1047, M918, M1073, BPrPr (1-28), MPG, Pep-1, MAP12, MAP17, GALA, p28, PreS2, VT5, Bac 7 [Bac (1-24)], PPR, PRR, SAP, SAP(E), CyLoP-1, gH 625, CPP-C, C105Y, Pep-7, and SG3.

15. The composition of claim 11, wherein the caspase inhibitor-cell penetrating peptide conjugate is a disulfide-linked Penetratin1-XBIR3.

16. The composition of claim 11, wherein the composition is formulated for injection, inhalation, or topical administration.

17. A method of preventing or decreasing inflammation by inhibiting a caspase-9 signaling pathway associated with inflammation or associated with the induction and/or exacerbation of diabetic macular edema (DME) and/or retinal vein occlusion (RVO) in a patient, the method comprising administering to a subject in need thereof an effective amount of a composition comprising a disulfide linked caspase inhibitor-cell penetrating peptide conjugate and a pharmaceutically acceptable carrier, wherein the caspase inhibitor-cell penetrating peptide conjugate has a concentration greater than 1 mM.

18. The method of claim 17, wherein the inflammation comprises appendicitis, bronchitis, bursitis, colitis, cystitis, dermatitis, encephalitis, gingivitis, meningitis, myelitis, nephritis, neuritis, periodontitis, pharyngitis, phlebitis, prostatitis, pulmonitis, rhinitis, sinusitis, tendonitis, tonsillitis, urethritis, vaginitis, or vasculitis.

19. The method of claim 17, wherein the caspase-9 signaling pathway does not involve modulation of VEGF-A levels, or induction of apoptosis in the cells expressing activated caspase-9, and the amount of the composition is therapeutically effective to prevent or decrease inflammation in one or more neuronal tissues by inhibiting the caspase-9 signaling pathway.

20. The method of claim 17, wherein the caspase-9 signaling pathway is associated with the induction and/or exacerbation of DME and/or RVO in a patient who has not responded to anti-vascular endothelial growth factor (VEGF) therapy.
